(19) United States
(12) Patent Application Publication
(10) Pub. No.: US 2025/0115671 A1
(43) Pub. Date: Apr. 10, 2025

(54) ANTI-CCR8 ANTIBODIES AND METHODS OF USE

(71) Applicant: BeiGene, Ltd., Grand Cayman (KY)

(72) Inventors: Ming FANG, Beijing (CN); Liu XUE, Beijing (CN); Hanzi SUN, Beijing (CN); Xiaoyan TANG, Beijing (CN); Ming JIANG, Beijing (CN); Xitao WANG, Beijing (CN); Yun CHEN, Shanghai (CN); Chichi HUANG, San Mateo, CA (US); Wenjie WANG, Beijing (CN); Jing ZHANG, Beijing (CN); Wenbo JIANG, Shanghai (CN)

(73) Assignee: BeiGene, Ltd., Grand Cayman (KY)

(21) Appl. No.: 18/930,933

(22) Filed: Oct. 29, 2024

Related U.S. Application Data
(63) Continuation of application No. PCT/CN2023/111215, filed on Aug. 4, 2023.

(30) Foreign Application Priority Data
Aug. 4, 2022 (WO) PCT/CN2022/110335

(51) Int. Cl. C07K 16/28; A61K 39/00; A61P 35/00

(52) U.S. Cl. CPC C07K 16/2866; A61P 35/00; A61K 2039/505; C07K 2317/34; C07K 2317/41; C07K 2317/52; C07K 2317/565; C07K 2317/732; C07K 2317/734; C07K 2317/92

(57) ABSTRACT

The present disclosure provides antibodies and antigen-binding fragments thereof that bind to human CCR8, a pharmaceutical composition comprising said antibody, and use of the antibody or the composition for treating a disease, such as cancer.

### CROSS-REFERENCE TO RELATED APPLICATIONS

This application is a bypass continuation of International Patent Application No. PCT/CN2023/111215, filed Aug. 4, 2023, which claims priority from International Patent Application No. PCT/CN2022/110335, filed Aug. 4, 2022. The contents of these applications are incorporated herein by reference in their entirety.

### SEQUENCE LISTING

The present application contains a Sequence Listing which is hereby incorporated by reference in its entirety. Said Sequence Listing XML file was created on Oct. 31, 2024, is 31 bytes in size, and is named 138881_1036_Sequence_Listing.xml.

### FIELD OF THE DISCLOSURE

Disclosed herein are antibodies or antigen-binding fragments thereof that bind to human CCR8, a composition comprising said antibody, as well as methods of use for the treatment of cancer.

### BACKGROUND

C—C motif chemokine receptor 8 (CCR8) is a seven-transmembrane G-protein coupled receptor (GPCR). Human CCR8 is composed of 355 amino acids, and its extracellular domains include a 35 amino acid (aa) N-terminus and three shorter extracellular loops (ECL1-3). The N-terminus and ECL2 are important for ligand binding and activation of CCR8 (Gadhe et al., J Biomol Struct Dyn. 2015; 33(11):2491-510; Barington L., et al., J Biol Chem. 2016 Jul. 29; 291(31):16208-20). Human CCR8 has four ligands including CCL1, CCL8, CCL16 and CCL18, while CCL1 is the primary ligand for CCR8 in T-regulatory cells (Tregs) and Th2 cells (Islam, S. A., et al., J Exp Med. 2013 Sep. 23; 210(10):1889-98; Barsheshet et al., Proc Natl Acad Sci USA. 2017 Jun. 6; 114(23):6086-6091; Sokol et al., 2018). CCR8 is normally expressed in fractions of Treg and Th2 cells in the blood (Schaerli et al., J Exp Med. 2004 May 3; 199(9):1265-75.; Soler et al., J Immunol. 2006 Nov. 15; 177(10):6940-51.), and also positive in thymus and spleen (Francesco Annunziato et al., J Exp Med. 2002 Aug. 5; 196(3):379-87.; Lee et al., J Immunol. 2007 Jan. 1; 178(1):301-11.; Thyagarajan et al., PLOS One. 2018 Jul. 19; 13(7):e0200765). Additionally, CCR8 is expressed in the skin T cells as well (Schaerli et al., J Exp Med. 2004 May 3; 199(9):1265-75.; Ebert et al., J Immunol. 2006 Apr. 1; 176(7):4331-6; Campbell et al., Cancer Res. 2021 Jun. 1; 81(11):2983-2994).

It has been well documented that CCR8 is markedly upregulated in tumor Tregs among different types of human cancers, including but not limited to head and neck, colorectal, lung, breast, bladder and esophageal (Plitas et al., Immunity. 2016 Nov. 15; 45(5):1122-1134; Magnuson et al., Proc Natl Acad Sci USA. 2018 Nov. 6; 115(45):E10672-E10681.; Wang et al., Cancer Immunol Immunother. 2020 September; 69(9):1855-1867; Van Damme et al., J Immunother Cancer. 2021 February; 9(2):e001749). High CCR8 expression also correlates with poor prognosis in breast, bladder and lung cancer patients (Plitas et al., Immunity. 2016 Nov. 15; 45(5):1122-1134; Alvisi et al., J Clin Invest. 2020 Jun. 1; 130(6):3137-3150.; Wang et al., Cancer Immunol Immunother. 2020 September; 69(9):1855-1867). CCR8 expression appears to mark an activated population of tumor Tregs as these cells have elevated activation markers and are more suppressive than CCR8 negative counterparts (Villarreal et al., Cancer Res. 2018 Sep. 15; 78(18):5340-5348; Wang et al., Nat Immunol. 2019 September; 20(9):1220-1230; Whiteside et al., Immunology. 2021 August; 163(4):512-520.). CCR8 activation by CCL1 can further potentiate the suppressive activity of CCR8+ Tregs by upregulating FOXp3, CCR8, CD39 and IL10 (Barsheshet et al., Proc Natl Acad Sci USA. 2017 Jun. 6; 114(23):6086-6091). Therefore, CCR8 is a promising therapeutic target to specifically target tumor Tregs, which play important suppressive roles in tumor microenvironment.

Recent studies in CCR8 null mice have shown that it is not required for tumor Treg recruitment or function; however, depletion of CCR8 Tregs using Fc intact but not mutated anti-mouse CCR8 antibodies have anti-tumor effect (Van Damme et al., J Immunother Cancer. 2021 February; 9(2):e001749; Bhatt et al., J Exp Med. 2021 Jun. 7; 218(6):e20201329; Campbell et al., Cancer Res. 2021 Jun. 1; 81(11):2983-2994; Kidani et al., Proc Natl Acad Sci USA. 2022 Feb. 15; 119(7):e2114282119). In addition to single agent activity, anti-mouse CCR8 antibodies have also demonstrated combo effect with anti-PD1 antibodies. In these studies, anti-mouse CCR8 antibodies do not induce toxicity in tumor bearing mice, and can spare spleen, thymus and skin Tregs (Villarreal et al., Cancer Res. 2018 Sep. 15; 78(18):5340-5348; Campbell et al., Cancer Res. 2021 Jun. 1; 81(11):2983-2994; Kidani et al., Proc Natl Acad Sci USA. 2022 Feb. 15; 119(7):e2114282119). CCR8−/− mice are also viable and fertile (Chung et al., J Immunol, 2003 Jan. 1; 170(1):581-7.; Yabe et al., Int Immunol. 2015 April; 27 (4):169-81). Together, these data indicate depletion of CCR8+ tumor Treg is a promising strategy and is unlikely to elicit deleterious effects. However, at this time, there are no CCR8 antibodies approved for use in the treatment of human cancer. Therefore, the anti-CCR8 antibodies as disclosed herein would be useful in the in the treatment of human cancers.

### SUMMARY OF THE DISCLOSURE

The present disclosure is directed to anti-CCR8 antibodies and antigen-binding fragments thereof that specifically bind CCR8.

In one embodiment, the disclosure provides for monoclonal antibodies that bind to human CCR8, or antigen-binding fragments thereof.

The present disclosure encompasses the following embodiments.

The antibody or antigen-binding fragment, wherein the antibody or antigen-binding fragment specifically binds human CCR8.

An antibody or antigen-binding fragment thereof, which specifically binds to human CCR8 (SEQ ID NO:1).

An antibody or antigen-binding fragment thereof which binds to human CCR8 comprising:
(i) a heavy chain variable region that comprises (a) a HCDR1 (Heavy Chain Complementarity Determining Region 1) of SEQ ID NO:4 (b) a HCDR2 of SEQ ID NO:5 and (c) a HCDR3 of SEQ ID NO:6 and a light chain variable region that comprises: (d) a LCDR1 (Light Chain Complementarity Determining Region 1) of SEQ ID NO:7, (e) a LCDR2 of SEQ ID NO:8, and (f) a LCDR3 of SEQ ID NO:9;
(ii) a heavy chain variable region that comprises (a) a HCDR1 (Heavy Chain Complementarity Determining Region 1) of SEQ ID NO:4 (b) a HCDR2 of SEQ ID NO:15 and (c) a HCDR3 of SEQ ID NO:6 and a light chain variable region that comprises: (d) a LCDR1 (Light Chain Complementarity Determining Region 1) of SEQ ID NO:7, (e) a LCDR2 of SEQ ID NO:8, and (f) a LCDR3 of SEQ ID NO:9; or
(iii) a heavy chain variable region that comprises (a) a HCDR1 (Heavy Chain Complementarity Determining Region 1) of SEQ ID NO:14 (b) a HCDR2 of SEQ ID NO:15 and (c) a HCDR3 of SEQ ID NO:6 and a light chain variable region that comprises: (d) a LCDR1 (Light Chain Complementarity Determining Region 1) of SEQ ID NO:7, (e) a LCDR2 of SEQ ID NO:8, and (f) a LCDR3 of SEQ ID NO:9.

The antibody or antigen-binding fragment comprising:
(i) a heavy chain variable region (VH) comprising an amino acid sequence at least 90, 91, 92, 93, 94, 95, 96, 97, 98 or 99% identical to SEQ ID NO:10, and a light chain variable region (VL) comprising an amino acid sequence at least 90, 91, 92, 93, 94, 95, 96, 97, 98 or 99% identical to SEQ ID NO: 11;
(ii) a heavy chain variable region (VH) comprising an amino acid sequence at least 90, 91, 92, 93, 94, 95, 96, 97, 98 or 99% identical to SEQ ID NO: 26, and a light chain variable region (VL) comprising an amino acid sequence at least 90, 91, 92, 93, 94, 95, 96, 97, 98 or 99% identical to SEQ ID NO: 23;
(iii) a heavy chain variable region (VH) comprising an amino acid sequence at least 90, 91, 92, 93, 94, 95, 96, 97, 98 or 99% identical to SEQ ID NO: 16, and a light chain variable region (VL) comprising an amino acid sequence at least 90, 91, 92, 93, 94, 95, 96, 97, 98 or 99% identical to SEQ ID NO: 17;
(iv) a heavy chain variable region (VH) comprising an amino acid sequence at least 90, 91, 92, 93, 94, 95, 96, 97, 98 or 99% identical to SEQ ID NO: 16, and a light chain variable region (VL) comprising an amino acid sequence at least 90, 91, 92, 93, 94, 95, 96, 97, 98 or 99% identical to SEQ ID NO: 20; or
(v) a heavy chain variable region (VH) comprising an amino acid sequence at least 90, 91, 92, 93, 94, 95, 96, 97, 98 or 99% identical to SEQ ID NO: 22, and a light chain variable region (VL) comprising an amino acid sequence at least 90, 91, 92, 93, 94, 95, 96, 97, 98 or 99% identical to SEQ ID NO: 23.

The antibody or antigen-binding fragment, wherein one, two, three, four, five, six, seven, eight, nine, or ten amino acids within SEQ ID NOs: 10 and 11, SEQ ID NOs: 26 and 23, SEQ ID NOs: 16 and 17, SEQ ID NOs: 16 and 20, or SEQ ID NOs: 22 and 23, have been inserted, deleted or substituted.

The antibody or antigen-binding fragment, that comprises:
(i) a heavy chain variable region (VH) that comprises SEQ ID NO:10, and a light chain variable region (VL) that comprises SEQ ID NO: 11;
(ii) a heavy chain variable region (VH) that comprises SEQ ID NO: 26, and a light chain variable region (VL) that comprises SEQ ID NO: 23;
(iii) a heavy chain variable region (VH) that comprises SEQ ID NO: 16, and a light chain variable region (VL) that comprises SEQ ID NO: 17;
(vi) a heavy chain variable region (VH) that comprises SEQ ID NO: 16, and a light chain variable region (VL) that comprises SEQ ID NO: 20; or
(v) a heavy chain variable region (VH) that comprises SEQ ID NO: 22, and a light chain variable region (VL) that comprises SEQ ID NO: 23.

The antibody or antigen-binding fragment, which is a monoclonal antibody, a chimeric antibody, a humanized antibody, a human engineered antibody, a single chain antibody (scFv), a Fab fragment, a Fab′ fragment, or a F(ab′)₂ fragment.

The antibody or antigen-binding fragment, wherein the antibody or antigen-binding fragment thereof has antibody dependent cellular cytotoxicity (ADCC) or complement dependent cytotoxicity (CDC).

The antibody or antigen-binding fragment, wherein the antibody or antigen-binding fragment thereof has reduced glycosylation or is hypofucosylated or afucosylated.

The antibody or antigen-binding fragment, wherein the antibody or antigen-binding fragment thereof comprises increased bisecting GlcNac structures.

The antibody or antigen-binding fragment, wherein the Fc domain is of an IgG1.

The antibody or antigen-binding fragment, which binds to epitopes comprising at least one, two, three, four, five, or six amnio acid residues at positions selected from number 26, 172, 177, 178, 266, and 269 of HuCCR8 amino acid sequence.

The antibody or antigen-binding fragment, which binds to epitopes comprising at least one, two or three amnio acid residue(s) at positions selected from number 172, 177, and 269 of HuCCR8 amino acid sequence.

The antibody or antigen-binding fragment, which binds to epitopes comprising at least one, two or three amnio acid residues at positions selected from number 26, 178, and 266 of HuCCR8 amino acid sequence.

The antibody or antigen-binding fragment, which binds to epitopes comprising (1) amino acid residue at position 20-30, (2) amino acid(s) at position 170-180, and (3) amino acid(s) at position 260-270 of HuCCR8 amino acid sequence.

The antibody or antigen-binding fragment, which binds to epitopes comprising (1) amino acid residue at position 26, (2) one, two or three amino acid(s) at position 172, 177 and 178, and (3) one or two amino acid(s) at position 266 and 269 of HuCCR8 amino acid sequence.

The antibody or antigen-binding fragment, which binds to epitopes comprising (1) one, two or three amino acid residues at positions in N-terminal, (2) one, two or three amino acid(s) at positions in ECL2 region, and (3) one, two or three amino acid(s) at positions in ECL3 region of HuCCR8 amino acid sequence.

A pharmaceutical composition comprising the antibody or antigen-binding fragment thereof, further comprising a pharmaceutically acceptable carrier.

A method of treating cancer comprising administering to a patient in need an effective amount of the antibody or antigen-binding fragment.

The method wherein the cancer is head and neck cancer, nasopharyngeal carcinoma, colon cancer, gastric cancer, breast cancer, pancreatic cancer, cervical cancer, bladder cancer, kidney cancer, colorectal cancer, esophageal cancer, ovarian cancer, liver cancer, non-small cell lung cancer, and small cell lung cancer.

The method wherein the antibody or antigen-binding fragment is administered in combination with another therapeutic agent.

The method, wherein the therapeutic agent is an immune checkpoint inhibitor.

The method, wherein the immune checkpoint inhibitor is an anti-PD1 antibody.

The method, wherein the anti-PD1 antibody is BGB-A317.

The method, wherein the immune checkpoint inhibitor is an anti-TIGIT antibody.

The method wherein the anti-TIGIT antibody is BGB-A1217.

The method, wherein the checkpoint inhibitor is BGB-A1217 and BGB-A317.

An isolated nucleic acid that encodes the antibody or antigen-binding fragment.

A vector comprising the nucleic acid.

A host cell comprising the nucleic acid or the vector.

A process for producing an antibody or antigen-binding fragment thereof comprising cultivating the host cell and recovering the antibody or antigen-binding fragment from the culture.

The antibody or antigen-binding fragment thereof, for use in the treatment or reducing the likelihood of: head and neck cancer, nasopharyngeal carcinoma, colon cancer, gastric cancer, breast cancer, pancreatic cancer, cervical cancer, bladder cancer, kidney cancer, colorectal cancer, esophageal cancer, ovarian cancer, liver cancer, non-small cell lung cancer, and small cell lung cancer.

In one embodiment, the antibody or an antigen-binding fragment thereof comprises one or more complementarity determining regions (CDRs) comprising an amino acid sequence selected from a group consisting of SEQ ID NO: 4, SEQ ID NO: 5, SEQ ID NO: 6, SEQ ID NO: 7, SEQ ID NO: 8, SEQ ID NO: 9, SEQ ID NO: 14, or SEQ ID NO: 15.

In another embodiment, the antibody or an antigen-binding fragment thereof comprises: (a) a heavy chain variable region comprising one or more complementarity determining regions (HCDRs) comprising an amino acid sequence selected from the group consisting of SEQ ID NO: 4, SEQ ID NO: 5, SEQ ID NO: 6, SEQ ID NO: 14, or SEQ ID NO: 15, and/or (b) a light chain variable region comprising one or more complementarity determining regions (LCDRs) comprising an amino acid sequence selected from the group consisting of SEQ ID NO: 7, SEQ ID NO: 8, or SEQ ID NO: 9.

In another embodiment, the antibody or an antigen-binding fragment thereof comprises: (a) a heavy chain variable region comprising three complementarity determining regions (HCDRs) which are HCDR1 comprising an amino acid sequence of SEQ ID NO: 4 or SEQ ID NO: 14, HCDR2 comprising an amino acid sequence of SEQ ID NO: 5 or SEQ ID NO: 15, and HCDR3 comprising an amino acid sequence of SEQ ID NO: 6, and/or (b) a light chain variable region comprising three complementarity determining regions (LCDRs) which are LCDR1 comprising an amino acid sequence of SEQ ID NO: 7, LCDR2 comprising an amino acid sequence of SEQ ID NO:8, and LCDR3 comprising an amino acid sequence of SEQ ID NO: 9.

In another embodiment, the antibody or an antigen-binding fragment thereof comprises: (a) a heavy chain variable region comprising three complementarity determining regions (HCDRs) which are HCDR1 comprising an amino acid sequence of SEQ ID NO: 4, HCDR2 comprising an amino acid sequence of SEQ ID NO: 5, and HCDR3 comprising an amino acid sequence of SEQ ID NO: 6; or HCDR1 comprising an amino acid sequence of SEQ ID NO: 14, HCDR2 comprising an amino acid sequence of SEQ ID NO: 15, and HCDR3 comprising an amino acid sequence of SEQ ID NO: 6; or HCDR1 comprising an amino acid sequence of SEQ ID NO: SEQ ID NO: 4, HCDR2 comprising an amino acid sequence of SEQ ID NO: 15, and HCDR3 comprising an amino acid sequence of SEQ ID NO: 6; and/or (b) a light chain variable region comprising three complementarity determining regions (LCDRs) which are LCDR1 comprising an amino acid sequence of SEQ ID NO: 7, LCDR2 comprising an amino acid sequence of SEQ ID NO: 8, and LCDR3 comprising an amino acid sequence of SEQ ID NO: 9.

In another embodiment, the antibody or the antigen-binding fragment of the present disclosure comprises: a heavy chain variable region comprising HCDR1 comprising an amino acid sequence SEQ ID NO: 4, HCDR2 comprising an amino acid sequence of SEQ ID NO: 5, and HCDR3 comprising an amino acid sequence of SEQ ID NO: 6; and a light chain variable region comprising LCDR1 comprising an amino acid sequence of SEQ ID NO: 7, LCDR2 comprising an amino acid sequence of SEQ ID NO: 8, and LCDR3 comprising an amino acid sequence of SEQ ID NO: 9.

In another embodiment, the antibody or the antigen-binding fragment of the present disclosure comprises: a heavy chain variable region comprising HCDR1 comprising an amino acid sequence SEQ ID NO: 14, HCDR2 comprising an amino acid sequence of SEQ ID NO: 15, and HCDR3 comprising an amino acid sequence of SEQ ID NO: 6; and a light chain variable region comprising LCDR1 comprising an amino acid sequence of SEQ ID NO: 7, LCDR2 comprising an amino acid sequence of SEQ ID NO: 8, and LCDR3 comprising an amino acid sequence of SEQ ID NO: 9.

In another embodiment, the antibody or the antigen-binding fragment of the present disclosure comprises: a heavy chain variable region comprising HCDR1 comprising an amino acid sequence SEQ ID NO: 4, HCDR2 comprising an amino acid sequence of SEQ ID NO: 15, and HCDR3 comprising an amino acid sequence of SEQ ID NO: 6; and a light chain variable region comprising LCDR1 comprising an amino acid sequence of SEQ ID NO: 7, LCDR2 comprising an amino acid sequence of SEQ ID NO: 8, and LCDR3 comprising an amino acid sequence of SEQ ID NO: 9.

In one embodiment, the antibody of the present disclosure or an antigen-binding fragment thereof comprises: (a) a heavy chain variable region comprising an amino acid sequence of SEQ ID NO: 10, SEQ ID NO: 16, SEQ ID NO: 22, or SEQ ID NO: 26 or an amino acid sequence at least 95%, 96%, 97%, 98% or 99% identical to any one of SEQ ID NO: 10, SEQ ID NO: 16, SEQ ID NO: 22, or SEQ ID NO: 26; and/or (b) a light chain variable region comprising an amino acid sequence of SEQ ID NO: 11, SEQ ID NO: 17, SEQ ID NO: 20, or SEQ ID NO: 23, or an amino acid sequence at least 95%, 96%, 97%, 98% or 99% identical to any one of SEQ ID NO: 11, SEQ ID NO: 17, SEQ ID NO: 20, or SEQ ID NO: 23.

In another embodiment, the antibody of the present disclosure or an antigen-binding fragment thereof comprises: (a) a heavy chain variable region comprising an amino acid sequence of SEQ ID NO: 10, SEQ ID NO: 16, SEQ ID NO: 22, or SEQ ID NO: 26, or an amino acid sequence comprising one, two, or three amino acid substitutions in the amino acid sequence of SEQ ID NO: 10, SEQ ID NO: 16, SEQ ID NO: 22, or SEQ ID NO: 26; and/or (b) a light chain variable region comprising an amino acid sequence of SEQ ID NO: 11, SEQ ID NO: 17, SEQ ID NO: 20, or SEQ ID NO: 23, or an amino acid sequence comprising one, two, three, four, or five amino acid substitutions in the amino acid of SEQ ID NO: 11, SEQ ID NO: 17, SEQ ID NO: 20, or SEQ ID NO: 23. In another embodiment, the amino acid substitutions are conservative amino acid substitutions.

In one embodiment, the antibody of the present disclosure or an antigen-binding fragment thereof comprises:
(a) a heavy chain variable region (VH) that comprises SEQ ID NO:10, and a light chain variable region (VL) that comprises SEQ ID NO: 11;
(b) a heavy chain variable region (VH) that comprises SEQ ID NO: 16, and a light chain variable region (VL) that comprises SEQ ID NO: 17;
(c) a heavy chain variable region (VH) that comprises SEQ ID NO: 16, and a light chain variable region (VL) that comprises SEQ ID NO: 20;
(d) a heavy chain variable region (VH) that comprises SEQ ID NO: 22, and a light chain variable region (VL) that comprises SEQ ID NO: 23; or
(e) a heavy chain variable region (VH) that comprises SEQ ID NO: 26, and a light chain variable region (VL) that comprises SEQ ID NO: 23.

In one embodiment, the antibody of the present disclosure is of IgG1, IgG2, IgG3, or IgG4 isotype. In a more specific embodiment, the antibody of the present disclosure comprises Fc domain of wild-type human IgG1 (also referred as human IgG1wt or huIgG1).

In one embodiment, the antibody of the present disclosure binds to human or cyno CCR8 with a binding affinity (EC50) of from 0.1 nM to 100 nM. In another embodiment, the antibody of the present disclosure binds to human CCR8 or Cyno CCR8 with a binding affinity (EC50) lower than 100 nM, 50 nM, 10 nM, 9 nM, 8 nM, 7 nM, 6 nM, 5 nM, 4 nM, 3 nM, 2 nM, 1 nM.

In another embodiment, the anti-human CCR8 antibody of the present disclosure shows a cross-species binding activity to cynomolgus monkey CCR8.

In one embodiment, antibodies of the present disclosure have strong Fc-mediated effector functions. The antibodies mediate antibody-dependent cellular cytotoxicity (ADCC) against CCR8 expressing target cells.

The present disclosure relates to isolated nucleic acids comprising nucleotide sequences encoding the amino acid sequence of the antibody or an antigen-binding fragment. In one embodiment, the isolated nucleic acid comprises a VH nucleotide sequence of SEQ ID NO: 12, SEQ ID NO: 18, SEQ ID NO: 24, or SEQ ID NO: 27 or a nucleotide sequence comprising at least 95%, 96%, 97%, 98% or 99% identity to SEQ ID NO: 12, SEQ ID NO: 18, SEQ ID NO: 24, or SEQ ID NO: 27, and encodes the VH region of the antibody or an antigen-binding fragment of the present disclosure. Alternatively or additionally, the isolated nucleic acid comprises a VL nucleotide sequence of SEQ ID NO: 13, SEQ ID NO: 19, SEQ ID NO: 21, or SEQ ID NO: 25 or a nucleotide sequence comprising at least 95%, 96%, 97%, 98% or 99% identity to SEQ ID NO: 13, SEQ ID NO: 19, SEQ ID NO: 21, or SEQ ID NO: 25 and encodes the VL region the antibody or an antigen-binding fragment of the present disclosure.

In another aspect, the present disclosure relates to a pharmaceutical composition comprising the CCR8 antibody or antigen-binding fragment thereof, and optionally a pharmaceutically acceptable excipient.

In yet another aspect, the present disclosure relates to a method of treating a disease in a subject, which comprises administering the CCR8 antibody or antigen-binding fragment thereof, or an CCR8 antibody pharmaceutical composition in a therapeutically effective amount to a subject in need thereof. In another embodiment the disease to be treated by the antibody or the antigen-binding fragment is cancer.

The current disclosure relates to use of the antibody or the antigen-binding fragment thereof, or an CCR8 antibody pharmaceutical composition for treating a disease, such as cancer.

### BRIEF DESCRIPTION OF THE DRAWINGS

FIG. 1A-B shows the immunogen human CCR8 protein sequence, comparison among human, monkey and mouse CCR8 and composition of human CCR8 N-terminus-Fc proteins. FIG. 1A shows the immunogen of human CCR8 protein sequence and comparison among human, monkey and mouse CCR8. FIG. 1B shows the composition of human CCR8-N-terminus-Fc protein.

FIG. 2 shows the comparison of blocking activity among representative chimeric anti-CCR8 antibodies.

FIG. 3 shows the comparison of CCR8 binding activity among representative chimeric anti-CCR8 antibodies using Jurkat human CCR8 overexpression cells.

FIG. 4A-I shows Glycan profiles of Ch305-AF, hu305-4F-21-AF and hu305-4F-2m-AF analyzed by Intact and reduced Mass Spectrometry. FIG. 4A-C shows the intact and reduced mass spectrometry results of Ch305-AF: FIG. 4A shows the intact mass result, FIG. 4B and FIG. 4C shows the reduced mass results of HC and LC, respectively. FIG. 4D-F shows the intact and reduced mass spectrometry results of hu305-4F-21-AF: FIG. 4D shows the intact mass result, FIG. 4E and FIG. 4F show the reduced mass results of HC and LC, respectively. FIG. 4G-I shows the intact and reduced mass spectrometry results of hu305-4F-2m-AF. FIG. 4G shows the intact mass result, FIG. 4H and FIG. 4I show the reduced mass results of HC and LC, respectively. These results together suggest the measured mass were consistent with the theoretical mass and no fucose related N-glycosylated mass was detected.

FIG. 5A-B shows the comparison of CCR8 binding activities of different humanized anti-CCR8 antibodies using Jurkat human CCR8 overexpression cells. FIG. 5A shows comparison among Ch305, hu305-4F-21 and hu305-4F-2m, while FIG. 5B shows comparison among Ch305, hu305-5W-3a and hu305-5P-3a.

FIG. 6A-B shows the comparison of binding activities to CCR8+ primary Treg (FIG. 6A), and CCR8 blocking activities among humanized Ch305 anti-CCR8 antibodies (FIG. 6B).

FIG. 7 shows the comparison of cyno CCR8 cross reactivity among different anti-CCR8 antibodies.

FIG. 8 shows the comparison of NK cell mediated cytotoxicity of different anti-CCR8 antibodies.

FIG. 9A-C shows the epitope identification of Ch305 by domain swapping and hu305-5W-3a by Alanine scanning. FIG. 9A shows the result of Ch305, and FIG. 9B shows the result of a reference antibody by domain swapping. FIG. 9C shows the result of hu305-5W-3a key epitope residues identified by Alanine scanning.

FIG. 10 shows the combination efficacy of anti-mouse CCR8 antibody with anti-mouse PD1 antibody in CT26 syngeneic mouse model.

FIG. 11 shows tumor Treg depletion effect of representative different anti-CCR8 antibodies in MC38 syngeneic model in human CCR8 KI mice.

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

The term “or” is used to mean, and is used interchangeably with, the term “and/or” unless the context clearly dictates otherwise.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “C—C motif chemokine receptor 8” or “CCR8” or “CD198” refers a cell receptor. The amino acid sequence of human CCR8 (SEQ ID NO: 1) can also be found at accession number P51685 (CCR8_HUMAN) or NP_005192.1. The amino acid sequence of Cynomolgus Monkey (“Cyno”) CCR8, (SEQ ID NO: 2) can also be found at accession number A0A8J8XUI3_MACFA or XP_015300839.1. The amino acid sequence of mouse CCR8, (SEQ ID NO: 3) can also be found at accession number P56484 (CCR8_MOUSE) or NP_031746.1.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human. Treating any disease or disorder refer in one aspect, to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder.

The term “subject” in the context of the present disclosure is a mammal, e.g., a primate, preferably a higher primate, e.g., a human (e.g., a patient having, or at risk of having, a disorder described herein).

The term “affinity” as used herein refers to the strength of interaction between antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA and IgY), or subclass (e.g., IgG1, IgG2, IgG3, IgG4, IgA1 and IgA2).

In some embodiments, the anti-CCR8 antibodies comprise at least one antigen-binding site, at least a variable region. In some embodiments, the anti-CCR8 antibodies comprise an antigen-binding fragment from an CCR8 antibody described herein. In some embodiments, the anti-CCR8 antibody is isolated or recombinant.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies comprising different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). See, Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196:901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but not limited to, Fab, Fab′, F(ab′)₂, and Fv fragments; diabodies; linear antibodies; single-chain antibody molecules, e.g., single chain Fv (ScFv); nanobodies and multi-specific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant (K_{D}, M)” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g. its binding affinity to CCR8. Specifically, common conservative substations of amino acids and are well known in the art.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include an anti-CCR8 antibody described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-CCR8 antibody is administered to the subject at the same time as, just before, or just after administration of an additional therapeutic agent. In certain embodiments, an anti-CCR8 antibody is administered as a co-formulation with an additional therapeutic agent.

### DETAILED DESCRIPTION

The present disclosure provides for antibodies, antigen-binding fragments, that specifically bind human CCR8. Furthermore, the present disclosure provides antibodies that have desirable pharmacokinetic characteristics and other desirable attributes, and thus can be used for reducing the likelihood of or treating cancer. The present disclosure further provides pharmaceutical compositions comprising the antibodies and methods of making and using such pharmaceutical compositions for the prevention and treatment of cancer and associated disorders.

### Anti-CCR8 Antibodies

The present disclosure provides for antibodies or antigen-binding fragments thereof that specifically bind to CCR8. Antibodies or antigen-binding fragments of the present disclosure include, but are not limited to, the antibodies or antigen-binding fragments thereof, generated as described, below.

The present disclosure provides antibodies or antigen-binding fragments that specifically bind to CCR8, wherein said antibodies or antibody fragments (e.g., antigen-binding fragments) comprise a VH domain comprising an amino acid sequence of SEQ ID NO: 10, SEQ ID NO: 16, SEQ ID NO: 22, or SEQ ID NO: 26 in Table 1. The present disclosure also provides antibodies or antigen-binding fragments that specifically bind CCR8, wherein said antibodies or antigen-binding fragments comprise a HCDR comprising an amino acid sequence of any one of the HCDRs listed in Table 1. In one aspect, the present disclosure provides antibodies or antigen-binding fragments that specifically bind to CCR8, wherein said antibodies comprise (or alternatively, consist of) one, two, three, or more HCDRs comprising an amino acid sequence of any of the HCDRs listed in Table 1.

The present disclosure provides for antibodies or antigen-binding fragments that specifically bind to CCR8, wherein said antibodies or antigen-binding fragments comprise a VL domain comprising an amino acid sequence of SEQ ID NO: 11, SEQ ID NO: 17, SEQ ID NO: 20, or SEQ ID NO: 23 (Table 1). The present disclosure also provides antibodies or antigen-binding fragments that specifically bind to CCR8, wherein said antibodies or antigen-binding fragments comprise a LCDR comprising an amino acid sequence of any one of the LCDRs listed in Table 1. In particular, the disclosure provides for antibodies or antigen-binding fragments that specifically bind to CCR8, said antibodies or antigen-binding fragments comprise (or alternatively, consist of) one, two, three or more LCDRs comprising an amino acid sequence of any of the LCDRs listed in Table 1.

Other antibodies or antigen-binding fragments thereof of the present disclosure include amino acids that have been changed, yet have at least 60%, 70%, 80%, 90%, 95% or 99% percent identity in the CDR regions with the CDR regions disclosed in Table 1. In some aspects, it includes amino acid changes wherein no more than 1, 2, 3, 4 or 5 amino acids have been changed in the CDR regions when compared with the CDR regions depicted in the sequence described in Table 1.

Other antibodies of the present disclosure include those where the amino acids or nucleic acids encoding the amino acids have been changed; yet have at least 60%, 70%, 80%, 90%, 95% or 99% percent identity to the sequences described in Table 1. In some aspects, it includes changes in the amino acid sequences wherein no more than 1, 2, 3, 4 or 5 amino acids have been changed in the variable regions when compared with the variable regions depicted in the sequence described in Table 1, while retaining substantially the same therapeutic activity.

The present disclosure also provides nucleic acid sequences that encode VH, VL, the full length heavy chain, and the full length light chain of the antibodies that specifically bind to CCR8. Such nucleic acid sequences can be optimized for expression in mammalian cells.

The sequences list of the present disclosure is provided below in Table 1.

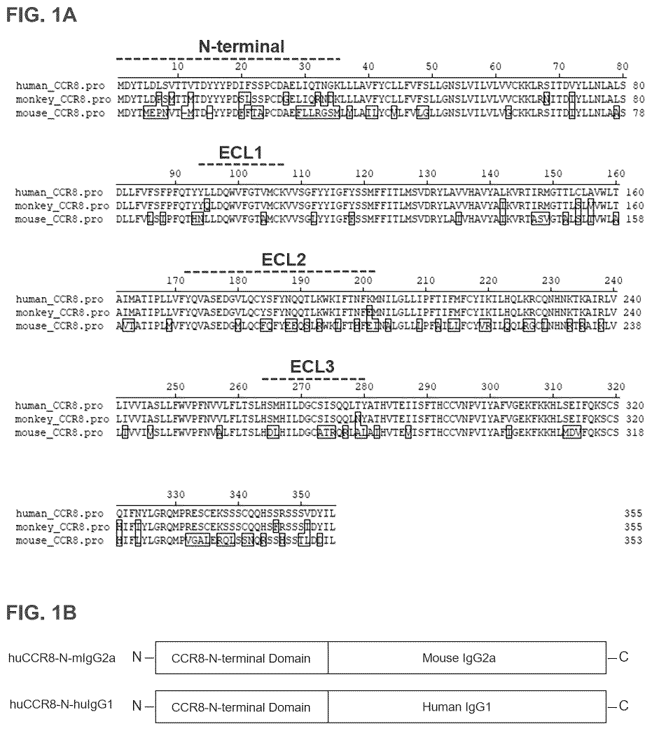

TABLE 1
Sequence List

| Antigen | SEQ ID NO: # | | SEQUENCE |
|---|---|---|---|
| Human | SEQ ID NO: 1 | | MDYTLDLSVTTVTDYYYPDIFSSPCDAE |
| CCR8 | | | LIQTNGKLLLAVFYCLLFVFSLLGNSLVIL |
| (same as | | | VLVVCKKLRSITDVYLLNLALSDLLFVFS |
| human_ | | | FPFQTYYLLDQWVFGTVMCKVVSGFYY |
| CCR8.pro in | | | IGFYSSMFFITLMSVDRYLAVVHAVYAL |
| FIG. 1A) | | | KVRTIRMGTTLCLAVWLTAIMATIPLLVF |
| | | | YQVASEDGVLQCYSFYNQQTLKWKIFT |
| | | | NFKMNILGLLIPFTIFMFCYIKILHQLKRC |
| | | | QNHNKTKAIRLVLIVVIASLLFWVPFNV |
| | | | VLFLTSLHSMHILDGCSISQQLTYATHVT |
| | | | EIISFTHCCVNPVIYAFVGEKFKKHLSEIF |
| | | | QKSCSQIFNYLGRQMPRESCEKSSSCQQ |
| | | | HSSRSSSVDYIL |
| Monkey | SEQ ID NO: 2 | | MDYTLDPSMTTMTDYYYPDSLSSPCDG |
| CCR8 | | | ELIQRNDKLLLAVFYCLLFVFSLLGNSLV |
| (same as | | | ILVLVVCKKLRNITDIYLLNLALSDLLFV |
| monkey_ | | | FSFPFQTYYQLDQWVFGTVMCKVVSGF |
| CCR8.pro | | | YYIGFYSSMFFITLMSVDRYLAVVHAVY |
| in FIG. 1A) | | | AIKVRTIRMGTTLSLVVWLTAIMATIPLL |
| | | | VFYQVASEDGVLQCYSFYNQQTLKWKI |
| | | | FTNFEMNILGLLIPFTIFMFCYIKILHQLK |
| | | | RCQNHNKTKAIRLVLIVVIASLLFWVPFN |
| | | | VVLFLTSLHSMHILDGCSISQQLNYATHV |
| | | | TEIISFTHCCVNPVIYAFVGEKFKKHLSEI |
| | | | FQKSCSHIFIYLGRQMPRESCEKSSSCQQ |
| | | | HSFRSSSIDYIL |
| Mouse | SEQ ID NO: 3 | | MDYTMEPNVTMTDYYPDFFTAPCDAEF |
| CCR8 | | | LLRGSMLYLAILYCVLFVLGLLGNSL VIL |
| (same as | | | VLVGCKKLRSITDIYLLNLAASDLLFVLS |
| mouse_ | | | IPFQTHNLLDQWVFGTAMCKVVSGLYYI |
| CCR8.pro in | | | GFFSSMFFITLMSVDRYLAIVHAVYAIKV |
| FIG. 1A) | | | RTASVGTALSLTVWLAAVTATIPLMVFY |
| | | | QVASEDGMLQCFQFYEEQSLRWKLFTH |
| | | | FEINALGLLLPFAILLFCYVRILQQLRGCL |
| | | | NHNRTRAIKLVLTVVIVSLLFWVPFNVA |
| | | | LFLTSLHDLHILDGCATRQRLALAIHVTE |
| | | | VISFTHCCVNPVIYAFIGEKFKKHLMDVF |
| | | | QKSCSHIFLYLGRQMPVGALERQLSSNQ |
| | | | RSSHSSTLDDIL |
| Antibody | SEQ ID NO: | | SEQUENCE |
| Ch305 | SEQ ID NO: 4 | HCDR1 | SYGVH |
| | | (Kabat) | |
| | SEQ ID NO: 5 | HCDR2 | VIWRGGTTDYNAAFMS |
| | | (Kabat) | |
| | SEQ ID NO: 6 | HCDR3 | NRVTTVVAPNFYAMDY |
| | | (Kabat) | |
| | SEQ ID NO: 7 | LCDR1 | KSSQSLLYSGNQKNYLA |
| | | (Kabat) | |
| | SEQ ID NO: 8 | LCDR2 | WASTRKS |
| | | (Kabat) | |
| | SEQ ID NO: 9 | LCDR3 | QQYYTYPLT |
| | | (Kabat) | |
| | SEQ ID NO: 10 | VH AA | QVQMKQSGPSLVQPSQSLSITCTVSGFSL |
| | | | TSYGVHWVRQSPGKGLEWLGVIWRGG |
| | | | TTDYNAAFMSRLSFTKDSSKSQVFFKLN |
| | | | SLQADDTAIYYCAKNRVTTVVAPNFYAM |
| | | | DYWGQGTSVTVSS |
| | SEQ ID NO: 11 | VL AA | DIVMTQSPSSLAVSVGEKVTMSCKSSQS |
| | | | LLYSGNQKNYLAWYQQKPGQSPKLLIY |
| | | | WASTRKSGVPDRLTGSGSGTDFTLTISSV |
| | | | KAEDLAAYYCQQYYTYPLTFGAGTRLEI |
| | | | K |
| | SEQ ID NO: 12 | VH DNA | CAGGTGCAGATGAAGCAGTCAGGACC |
| | | | TAGCCTAGTGCAGCCCTCACAGAGCCT |
| | | | GTCCATAACCTGCACAGTCTCTGGTTTC |
| | | | TCATTAACTAGCTATGGGGTACACTGGG |
| | | | TTCGCCAGTCTCCAGGAAAGGGTCTGG |
| | | | AGTGGCTGGGAGTGATATGGAGAGGTG |
| | | | GAACCACAGACTACAATGCAGCTTTCA |
| | | | TGTCCAGACTGAGCTTCACCAAGGACA |
| | | | GCTCCAAGAGCCAAGTTTTCTTTAAAT |
| | | | TGAACAGTCTGCAAGCTGATGACACTG |
| | | | CCATATACTACTGTGCCAAGAATCGGGT |
| | | | AACTACGGTTGTAGCCCCAAATTTCTAT |
| | | | GCTATGGACTACTGGGGTCAAGGAACC |
| | | | TCAGTCACCGTCTCGAGC |
| | SEQ ID NO: 13 | VL DNA | GACATTGTGATGACCCAGTCTCCATCCT |
| | | | CCCTAGCTGTGTCAGTTGGAGAGAAGG |
| | | | TTACTATGAGCTGCAAGTCCAGTCAGA |
| | | | GCCTTTTATATAGTGGCAATCAAAAGAA |
| | | | CTACTTGGCCTGGTACCAGCAGAAACC |
| | | | AGGGCAGTCTCCCAAACTGCTGATTTA |
| | | | CTGGGCATCCACTAGGAAATCTGGGGT |
| | | | CCCTGATCGCCTCACAGGCAGTGGATC |
| | | | TGGGACAGATTTCACTCTCACCATCAG |
| | | | CAGTGTGAAGGCTGAAGACCTGGCAG |
| | | | CTTATTACTGTCAGCAATATTATACCTAT |
| | | | CCGCTCACGTTCGGTGCTGGGACCAGA |
| | | | CTGGAAATAAAA |
| hu305-4F- | SEQ ID NO: 14 | HCDR1 | VYGVH |
| 2m | | (Kabat) | |
| | SEQ ID NO: 15 | HCDR2 | VIWRGGTTDYNPSLKS |
| | | (Kabat) | |
| | SEQ ID NO: 6 | HCDR3 | NRVTTVVAPNFYAMDY |
| | | (Kabat) | |
| | SEQ ID NO: 7 | LCDR1 | KSSQSLLYSGNQKNYLA |
| | | (Kabat) | |
| | SEQ ID NO: 8 | LCDR2 | WASTRKS |
| | | (Kabat) | |
| | SEQ ID NO: 9 | LCDR3 | QQYYTYPLT |
| | | (Kabat) | |
| | SEQ ID NO: 16 | VH AA | QVQLQESGPGLVKPSQTLSLTCTVSGFSL |
| | | | AVYGVHWVRQPPGKGLEWLGVIWRG |
| | | | GTTDYNPSLKSRLTFSKDSSKSQVSLKL |
| | | | SSVTAADTAVYYCAKNRVTTVVAPNFY |
| | | | AMDYWGQGTLVTVSS |
| | SEQ ID NO: 17 | VL AA | DIVMTQSPDSLAVSLGERVTINCKSSQSL |
| | | | LYSGNQKNYLAWYQQKPGQSPKLLIYW |
| | | | ASTRKSGVPDRFSGSGSGTDFTLTISSLQ |
| | | | AEDVAAYYCQQYYTYPLTFGQGTRLEIK |
| | SEQ ID NO: 18 | VH DNA | CAAGTGCAGCTGCAAGAGAGCGGCCC |
| | | | CGGCCTGGTGAAGCCTAGCCAGACCCT |
| | | | GAGCCTGACCTGCACCGTGAGCGGCTT |
| | | | CAGCCTGGCAGTCTACGGCGTGCACTG |
| | | | GGTGAGACAGCCCCCCGGCAAGGGCC |
| | | | TGGAGTGGCTGGGCGTGATCTGGAGAG |
| | | | GCGGCACCACCGACTACAACCCCTCCC |
| | | | TCAAGAGCAGACTGACCTTCAGCAAG |
| | | | GACAGCAGCAAGAGCCAAGTGTCCCT |
| | | | CAAGCTGAGCAGCGTGACCGCCGCCG |
| | | | ACACCGCCGTGTACTACTGCGCCAAGA |
| | | | ACAGAGTGACCACCGTGGTGGCCCCC |
| | | | AACTTCTACGCCATGGACTACTGGGGC |
| | | | CAAGGCACCCTGGTGACCGTGAGCAG |
| | | | C |
| | SEQ ID NO: 19 | VL DNA | GACATCGTGATGACACAGAGCCCCGAC |
| | | | AGCCTGGCCGTGAGCCTGGGCGAGAG |
| | | | AGTCACCATCAACTGCAAGAGCAGCCA |
| | | | AAGCCTGCTGTACAGCGGCAATCAGAA |
| | | | GAACTACCTGGCCTGGTATCAGCAGAA |
| | | | GCCCGGGCAGAGCCCCAAGCTGCTGAT |
| | | | CTACTGGGCCTCCACAAGAAAGAGCG |
| | | | GCGTGCCCGACAGATTCAGCGGCAGCG |
| | | | GCAGCGGCACCGACTTCACCCTGACCA |
| | | | TCAGCAGCCTGCAAGCCGAGGACGTG |
| | | | GCCGCGTACTACTGTCAGCAGTACTAC |
| | | | ACCTACCCCCTGACCTTCGGCCAAGGC |
| | | | ACAAGACTGGAGATCAAG |
| hu305-4F- | SEQ ID NO: 14 | HCDR1 | VYGVH |
| 2l | | (Kabat) | |
| | SEQ ID NO: 15 | HCDR2 | VIWRGGTTDYNPSLKS |
| | | (Kabat) | |
| | SEQ ID NO: 6 | HCDR3 | NRVTTVVAPNFYAMDY |
| | | (Kabat) | |
| | SEQ ID NO: 7 | LCDR1 | KSSQSLLYSGNQKNYLA |
| | | (Kabat) | |
| | SEQ ID NO: 8 | LCDR2 | WASTRKS |
| | | (Kabat) | |
| | SEQ ID NO: 9 | LCDR3 | QQYYTYPLT |
| | | (Kabat) | |
| | SEQ ID NO: 16 | VH AA | QVQLQESGPGLVKPSQTLSLTCTVSGFSL |
| | | | AVYGVHWVRQPPGKGLEWLGVIWRG |
| | | | GTTDYNPSLKSRLTFSKDSSKSQVSLKL |
| | | | SSVTAADTAVYYCAKNRVTTVVAPNFY |
| | | | AMDYWGQGTLVTVSS |
| | SEQ ID NO: 20 | VL AA | DIVMTQSPDSLAVSLGERVTMNCKSSQS |
| | | | LLYSGNQKNYLAWYQQKPGQSPKLLIY |
| | | | WASTRKSGVPDRFSGSGSGTDFTLTISSL |
| | | | QAEDVAAYYCQQYYTYPLTFGQGTRLE |
| | | | IK |
| | SEQ ID NO: 18 | VH DNA | CAAGTGCAGCTGCAAGAGAGCGGCCC |
| | | | CGGCCTGGTGAAGCCTAGCCAGACCCT |
| | | | GAGCCTGACCTGCACCGTGAGCGGCTT |
| | | | CAGCCTGGCAGTCTACGGCGTGCACTG |
| | | | GGTGAGACAGCCCCCCGGCAAGGGCC |
| | | | TGGAGTGGCTGGGCGTGATCTGGAGAG |
| | | | GCGGCACCACCGACTACAACCCCTCCC |
| | | | TCAAGAGCAGACTGACCTTCAGCAAG |
| | | | GACAGCAGCAAGAGCCAAGTGTCCCT |
| | | | CAAGCTGAGCAGCGTGACCGCCGCCG |
| | | | ACACCGCCGTGTACTACTGCGCCAAGA |
| | | | ACAGAGTGACCACCGTGGTGGCCCCC |
| | | | AACTTCTACGCCATGGACTACTGGGGC |
| | | | CAAGGCACCCTGGTGACCGTGAGCAG |
| | | | C |
| | SEQ ID NO: 21 | VL DNA | GACATCGTGATGACACAGAGCCCCGAC |
| | | | AGCCTGGCCGTGAGCCTGGGCGAGAG |
| | | | AGTCACCATGAACTGCAAGAGCAGCC |
| | | | AAAGCCTGCTGTACAGCGGCAATCAGA |
| | | | AGAACTACCTGGCCTGGTATCAGCAGA |
| | | | AGCCCGGGCAGAGCCCCAAGCTGCTG |
| | | | ATCTACTGGGCCTCCACAAGAAAGAGC |
| | | | GGCGTGCCCGACAGATTCAGCGGCAGC |
| | | | GGCAGCGGCACCGACTTCACCCTGACC |
| | | | ATCAGCAGCCTGCAAGCCGAGGACGT |
| | | | GGCCGCGTACTACTGTCAGCAGTACTA |
| | | | CACCTACCCCCTGACCTTCGGCCAAGG |
| | | | CACAAGACTGGAGATCAAG |
| hu305-5W- | SEQ ID NO: 14 | HCDR1 | VYGVH |
| 3a | | (Kabat) | |
| | SEQ ID NO: 15 | HCDR2 | VIWRGGTTDYNPSLKS |
| | | (Kabat) | |
| | SEQ ID NO: 6 | HCDR3 | NRVTTVVAPNFYAMDY |
| | | (Kabat) | |
| | SEQ ID NO: 7 | LCDR1 | KSSQSLLYSGNQKNYLA |
| | | (Kabat) | |
| | SEQ ID NO: 8 | LCDR2 | WASTRKS |
| | | (Kabat) | |
| | SEQ ID NO: 9 | LCDR3 | QQYYTYPLT |
| | | (Kabat) | |
| | SEQ ID NO: 22 | VH AA | QVQLKQSGPGLVKPSETLSLTCTVSGFSL |
| | | | AVYGVHWVRQPPGKGLEWLGVIWRG |
| | | | GTTDYNPSLKSRLTFSKDSSKSQVSLKL |
| | | | SSVTAADTAVYYCAKNRVTTVVAPNFY |
| | | | AMDYWGQGTLVTVSS |
| | SEQ ID NO: 23 | VL AA | DIVMTQSPDSLAVSLGERATINCKSSQSL |
| | | | LYSGNQKNYLAWYQQKPGQPPKLLIY |
| | | | WASTRKSGVPDRFSGSGSGTDFTLTISSL |
| | | | KAEDVAVYYCQQYYTYPLTFGQGTRLE |
| | | | IK |
| | SEQ ID NO: 24 | VH DNA | CAAGTGCAGCTGAAACAGAGCGGCCC |
| | | | CGGCCTGGTGAAGCCTAGCGAGACCCT |
| | | | GAGCCTGACCTGCACCGTGAGCGGCTT |
| | | | CAGCCTGGCAGTCTACGGCGTGCACTG |
| | | | GGTGAGACAGCCCCCCGGCAAGGGCC |
| | | | TGGAGTGGCTGGGCGTGATCTGGAGAG |
| | | | GCGGCACCACCGACTACAACCCCTCCC |
| | | | TCAAGAGCAGACTGACCTTCAGCAAG |
| | | | GACAGCAGCAAGAGCCAAGTGTCCCT |
| | | | CAAGCTGAGCAGCGTGACCGCCGCCG |
| | | | ACACCGCCGTGTACTACTGCGCCAAGA |
| | | | ACAGAGTGACCACCGTGGTGGCCCCC |
| | | | AACTTCTACGCCATGGACTACTGGGGC |
| | | | CAAGGCACCCTGGTGACCGTGAGCAG |
| | | | C |
| | SEQ ID NO: 25 | VL DNA | GACATCGTGATGACACAGAGCCCCGAC |
| | | | AGCCTGGCCGTGAGCCTGGGCGAGAG |
| | | | AGCCACCATCAACTGCAAGAGCAGCC |
| | | | AAAGCCTGCTGTACAGCGGCAATCAGA |
| | | | AGAACTACCTGGCCTGGTATCAGCAGA |
| | | | AGCCCGGGCAGCCCCCCAAGCTGCTG |
| | | | ATCTACTGGGCCTCCACAAGAAAGAGC |
| | | | GGCGTGCCCGACAGATTCAGCGGCAGC |
| | | | GGCAGCGGCACCGACTTCACCCTGACC |
| | | | ATCAGCAGCCTGAAAGCCGAGGACGT |
| | | | GGCCGTGTACTACTGTCAGCAGTACTA |
| | | | CACCTACCCCCTGACCTTCGGCCAAGG |
| | | | CACAAGACTGGAGATCAAG |
| hu305-5P- | SEQ ID NO: 4 | HCDR1 | SYGVH |
| 3a | | (Kabat) | |
| | SEQ ID NO: 15 | HCDR2 | VIWRGGTTDYNPSLKS |
| | | (Kabat) | |
| | SEQ ID NO: 6 | HCDR3 | NRVTTVVAPNFYAMDY |
| | | (Kabat) | |
| | SEQ ID NO: 7 | LCDR1 | KSSQSLLYSGNQKNYLA |
| | | (Kabat) | |
| | SEQ ID NO: 8 | LCDR2 | WASTRKS |
| | | (Kabat) | |
| | SEQ ID NO: 9 | LCDR3 | QQYYTYPLT |
| | | (Kabat) | |
| | SEQ ID NO: 26 | VH AA | QVQLKQSGPGLVKPSETLSLTCTVSGFSL |
| | | | TSYGVHWVRQPPGKGLEWLGVIWRGG |
| | | | TTDYNPSLKSRVTFSKDSSKNQVSLKLSS |
| | | | VTAADTAVYYCAKNRVTTVVAPNFYAM |
| | | | DYWGQGTLVTVSS |
| | SEQ ID NO: 23 | VL AA | DIVMTQSPDSLAVSLGERATINCKSSQSL |
| | | | LYSGNQKNYLAWYQQKPGQPPKLLIY |
| | | | WASTRKSGVPDRFSGSGSGTDFTLTISSL |
| | | | KAEDVAVYYCQQYYTYPLTFGQGTRLE |
| | | | IK |
| | SEQ ID NO: 27 | VH DNA | CAAGTGCAGCTGAAACAGAGCGGCCC |
| | | | CGGCCTGGTGAAGCCTAGCGAGACCCT |
| | | | GAGCCTGACCTGCACCGTGAGCGGCTT |
| | | | CAGCCTGACAAGCTACGGCGTGCACTG |
| | | | GGTGAGACAGCCCCCCGGCAAGGGCC |
| | | | TGGAGTGGCTGGGCGTGATCTGGAGAG |
| | | | GCGGCACCACCGACTACAACCCCTCCC |
| | | | TCAAGAGCAGAGTGACCTTCAGCAAG |
| | | | GACTCCAGCAAGAACCAAGTGTCCCTC |
| | | | AAGCTGAGCAGCGTGACCGCCGCCGA |
| | | | CACCGCCGTGTACTACTGCGCCAAGAA |
| | | | CAGAGTGACCACCGTGGTGGCCCCCA |
| | | | ACTTCTACGCCATGGACTACTGGGGCC |
| | | | AAGGCACCCTGGTGACCGTGAGCAGC |
| | SEQ ID NO: 25 | VL DNA | GACATCGTGATGACACAGAGCCCCGAC |
| | | | AGCCTGGCCGTGAGCCTGGGCGAGAG |
| | | | AGCCACCATCAACTGCAAGAGCAGCC |
| | | | AAAGCCTGCTGTACAGCGGCAATCAGA |
| | | | AGAACTACCTGGCCTGGTATCAGCAGA |
| | | | AGCCCGGGCAGCCCCCCAAGCTGCTG |
| | | | ATCTACTGGGCCTCCACAAGAAAGAGC |
| | | | GGCGTGCCCGACAGATTCAGCGGCAGC |
| | | | GGCAGCGGCACCGACTTCACCCTGACC |
| | | | ATCAGCAGCCTGAAAGCCGAGGACGT |
| | | | GGCCGTGTACTACTGTCAGCAGTACTA |
| | | | CACCTACCCCCTGACCTTCGGCCAAGG |
| | | | CACAAGACTGGAGATCAAG |
| Antigen | SEQ ID | | SEQUENCE |
| huCCR8- | SEQ ID NO: 28 | | MDYTLDLSVTTVTDYYYPDIFSSPSDAE |
| N-mIgG2a | | | LIQTNGKGGGGSCPPCKCPAPNLLGGPS |
| | | | VFIFPPKIKDVLMISLSPIVTCVVVDVSED |
| | | | DPDVQISWFVNNVEVHTAQTQTHREDY |
| | | | NSTLRVVSALPIQHQDWMSGKEFKCKV |
| | | | NNKDLPAPIERTISKPKGSVRAPQVYVLP |
| | | | PPEEEMTKKQVTLTCMVTDFMPEDIYVE |
| | | | WTNNGKTELNYKNTEPVLDSDGSYFMY |
| | | | SKLRVEKKNWVERNSYSCSVVHEGLHN |
| | | | HHTTKSFSRTPGK |
| huCCR8- | SEQ ID NO: 29 | | MDYTLDLSVTTVTDYYYPDIFSSPSDAE |
| N-huIgG1 | | | LIQTNGKGGGGSCPPCPAPELLGGPSVFL |
| | | | FPPKPKDTLMISRTPEVTCVVVDVSHEDP |
| | | | EVKFNWYVDGVEVHNAKTKPREEQYN |
| | | | STYRVVSVLTVLHQDWLNGKEYKCKVS |
| | | | NKALPAPIEKTISKAKGQPREPQVYTLPP |
| | | | SRDELTKNQVSLTCLVKGFYPSDIAVEWE |
| | | | SNGQPENNYKTTPPVLDSDGSFFLYSKLT |
| | | | VDKSRWQQGNVFSCSVMHEALHNHYT |
| | | | QKSLSLSPGK |

Identification of Epitopes and Antibodies that Bind to the Same Epitope

The present disclosure provides antibodies and antigen-binding fragments thereof that bind to an epitope of human CCR8. In certain aspects the antibodies and antigen-binding fragments can bind to the same epitope of CCR8.

The present disclosure also provides for antibodies and antigen-binding fragments thereof that bind to the same epitope as do the anti-CCR8 antibodies described in Table 1. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CCR8 demonstrates that the test antibody can compete with that antibody or antigen-binding fragments thereof for binding to CCR8. Such an antibody can, without being bound to any one theory, bind to the same or a related (e.g., a structurally similar or spatially proximal) epitope on CCR8 as the antibody or antigen-binding fragments thereof with which it competes. In a certain aspect, the antibody that binds to the same epitope on CCR8 as the antibodies or antigen-binding fragments thereof of the present disclosure is a human or humanized monoclonal antibody. Such human or humanized monoclonal antibodies can be prepared and isolated as described herein.

### Further Alteration of the Framework of Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered Clq binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001).

In still another aspect, the glycosylation of an antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al., describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

### CCR8 Antibody Production

Anti-CCR8 antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 10, SEQ ID NO: 16, SEQ ID NO: 22, or SEQ ID NO: 26. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting SEQ ID NO: 11, SEQ ID NO: 17, SEQ ID NO: 20, or SEQ ID NO: 23.

The polynucleotides of the present disclosure can encode the variable region sequence of an anti-CCR8 antibody. They can also encode both a variable region and a constant region of the antibody. Some of the polynucleotide sequences encode a polypeptide that comprises variable regions of both the heavy chain and the light chain of one of the exemplified anti-CCR8 antibodies.

Also provided in the present disclosure are expression vectors and host cells for producing the anti-CCR8 antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-CCR8 antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-CCR8 antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-CCR8 antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-CCR8 polypeptides. Insect cells in combination with baculovirus vectors can also be used.

In other aspects, mammalian host cells are used to express and produce the anti-CCR8 polypeptides of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Methods of Detection and Diagnosis

The antibodies or antigen-binding fragments of the present disclosure are useful in a variety of applications including, but not limited to, methods for the detection of CCR8. In one aspect, the antibodies or antigen-binding fragments are useful for detecting the presence of CCR8 in a biological sample. The term “detecting” as used herein includes quantitative or qualitative detection. In certain aspects, a biological sample comprises a cell or tissue. In other aspects, such tissues include normal and/or cancerous tissues that express CCR8 at higher levels relative to other tissues.

In one aspect, the present disclosure provides a method of detecting the presence of CCR8 in a biological sample. In certain aspects, the method comprises contacting the biological sample with an anti-CCR8 antibody under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of CCR8. In certain aspects, the method comprises contacting a test cell with an anti-CCR8 antibody; determining the level of expression (either quantitatively or qualitatively) of CCR8 expressed by the test cell by detecting binding of the anti-CCR8 antibody to the CCR8 polypeptide; and comparing the level of expression by the test cell with the level of CCR8 expression in a control cell (e.g., a normal cell of the same tissue origin as the test cell or a non-CCR8 expressing cell), wherein a higher level of CCR8 expression in the test cell as compared to the control cell indicates the presence of a disorder associated with expression of CCR8.

### Methods of Treatment

The antibodies or antigen-binding fragments of the present disclosure are useful in a variety of applications including, but not limited to, methods for the treatment of a CCR8-associated disorder or disease. In one aspect, the CCR8-associated disorder or disease is a cancer.

In one aspect, the present disclosure provides a method of treating cancer. In certain aspects, the method comprises administering to a patient in need an effective amount of an anti-CCR8 antibody or antigen-binding fragment. The cancer can include, without limitation, head and neck cancer, nasopharyngeal carcinoma, colon cancer, gastric cancer, breast cancer, pancreatic cancer, cervical cancer, bladder cancer, kidney cancer, colorectal cancer, esophageal cancer, ovarian cancer, liver cancer, non-small cell lung cancer, and small cell lung cancer. More specifically, the cancer can include head and neck squamous cell carcinoma, nasopharyngeal carcinoma, colorectal carcinoma with high microsatellite instability, lung adenocarcinoma, lung squamous cell carcinoma, gastric/stomach adenocarcinoma, triple-negative breast cancer, human epidermal growth factor receptor 2 (her2)+ breast cancer, pancreatic adenocarcinoma, cervical squamous cell carcinoma and endocervical adenocarcinoma, urothelial bladder carcinoma, renal cell carcinoma (kidney renal clear cell carcinoma), colorectal cancer with microsatellite stable (CRC_MSS), esophageal squamous cell carcinoma, esophageal adenocarcinoma, progesterone/estrogen receptor positive breast cancer, liver hepatocellular carcinoma.

The antibody or antigen-binding fragment as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antigen-binding fragment of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 100 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses can be administered intermittently, e.g., every week or every three weeks (e.g., such that the patient receives from about two to about twenty, or e.g., about six doses of the antibody). An initial higher loading dose, followed by one or more lower doses can be administered. However, other dosage regimens can be useful. The progress of this therapy is easily monitored by conventional techniques and assays.

### Combination Therapy

In one aspect, CCR8 antibodies of the present disclosure can be used in combination with other therapeutic agents. Other therapeutic agents that can be used with the CCR8 antibodies of the present disclosure include: but are not limited to, a chemotherapeutic agent (e.g., paclitaxel or a paclitaxel agent; (e.g., Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672).

Anti-CCR8 antibodies of the present disclosure can be used in combination with other therapeutics, for example, other immune checkpoint antibodies. Such immune checkpoint antibodies can include anti-PD1 antibodies. Anti-PD1 antibodies can include, without limitation, antibodies disclosed in U.S. Pat. No. 8,735,553. Pembrolizumab (formerly MK-3475), as disclosed by Merck, is a humanized lgG4-K immunoglobulin with a molecular weight of about 149 kDa, which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb is a fully human lgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

Other immune checkpoint antibodies for combination with anti-CCR8 antibodies can include anti-TIGIT antibodies. Such anti-TIGIT antibodies can include without limitation, anti-TIGIT antibodies disclosed in WO2019/129261.

### Pharmaceutical Compositions and Formulations

Also provided are compositions, including pharmaceutical formulations, comprising an anti-CCR8 antibody or antigen-binding fragment thereof, or polynucleotides comprising sequences encoding an anti-CCR8 antibody or antigen-binding fragment. In certain embodiments, compositions comprise one or more antibodies or antigen-binding fragments that bind to CCR8, or one or more polynucleotides comprising sequences encoding one or more antibodies or antigen-binding fragments that bind to CCR8. These compositions can further comprise suitable carriers, such as pharmaceutically acceptable excipients including buffers, which are well known in the art.

Pharmaceutical formulations of an anti-CCR8 antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g., films, or microcapsules.

The formulations to be used for in vivo administration are generally sterile. Sterility can be readily accomplished, e.g., by filtration through sterile filtration membranes.

### EXAMPLES

### Example 1. Generation of Mouse Anti-CCR8 Antibodies

### Mouse Immunization

To generate antibodies against CCR8, a total of 50 adult female mice from different strains (BALB/c, C57BL/6, SJL, and MRL/lpr) were immunized with different combinations of immunogens comprising human CCR8 expression plasmid (huCCR8 DNA), L929 cells overexpressing human CCR8 (L929-huCCR8), and L929 cells overexpressing cynomolgus CCR8 (L929-cynoCCR8). Human, cynomolgus and mouse CCR8 protein sequences are listed as SEQ ID NO: 1 to 3 in Table 1 respectively, the comparison among human, monkey and mouse CCR8 and composition of human CCR8 N-terminus-Fc proteins are shown in FIG. 1. The human CCR8 expression plasmid (huCCR8 DNA) (50 μg per mouse) was intramuscularly delivered by in vivo electroporation using an EPT-I delivery device (TERESA, Shanghai, China) as described previously (Jiang et al., J Virol. 2017 Apr. 13; 91(9):e02052-16). L929-huCCR8 and L929-cynoCCR8 cells (5×10⁶ cells per mouse) were intraperitoneally injected. Typically, 5-7 immunizations with three-week interval were performed to induce potent anti-CCR8 humoral immune responses. At day 14 post each immunization, blood was collected and serum titer against 293T cells overexpressing human CCR8 (293T-huCCR8) and 293T cells overexpressing cynomolgus CCR8 (293T-cynoCCR8) was determined by FACS. Animals with sufficient levels of anti-CCR8 serum antibodies were selected for final boost.

### Plasma Cell Screening by the Beacon Optofluidic System

At 3-5 days post final boost, spleens were collected and mashed into single cell suspensions. Plasma cells were isolated with mouse CD138 positive selection kit (STEMCELL) according to the manufacturer's instructions. Enriched plasma cells with a density of 6.25×10⁶/ml were imported into the channel and penned into the NanoPen chambers of OptoSelect 14K Chip™ (Berkeley Lights) according to the manufacturer's instructions. To screen huCCR8 specific plasma cells, 293T-huCCR8 cells with a density of 1×10⁸/ml and Alexa Fluor 647 goat anti-mouse IgG secondary antibody (Jackson ImmunoResearch) with a concentration of 5 μg/ml were imported into the channel. Following import, the freeze valve was turned on and the exposure time of CY5 channel for Alexa Fluor 647 fluorophore was set to 3000 ms. Bloom-like positive signals were captured by time lapse imaging with settings of 6-minute period and 10 cycles. After completion of huCCR8 specific plasma cell screening with 293T-huCCR8 cells, cynoCCR8 specific plasma cells were screened with 293T-cynoCCR8 cells. Plasma cells showed positive signals against both 293T-huCCR8 and 293T-cynoCCR8 cells were individually exported into 96-well plates filled with lysis buffer.

### Example 2. Antibody VH and VL Gene Cloning, Sequencing, and Chimeric Antibody Expression

First-strand cDNA was synthesized, and total cDNA was amplified with Opto Plasma B Discovery cDNA Synthesis Kit™ (Berkeley Lights) according to the manufacturer's instructions. Antibody VH and VL genes were amplified with Opto Plasma B Discovery Sanger Prep Kit™ (Berkeley Lights) according to the manufacturer's instructions. The amplified VH and VL genes were cloned into mammalian expression vector containing human IgG1 and kappa chain constant region genes, respectively. The amino acid sequences of the 3 HCDRs, 3 LCDRs, VH and VL and the DNA sequences of the VH and VL for representative antibody Ch305 from clone PBG04-305 (also referred as clone 305) are listed in Table 1 as SEQ ID NO:4 to 13. Chimeric antibodies were expressed by Expi293™ cells and purified by affinity chromatography.

### Example 3. Determination of the Binding Affinity and Specificity of Anti-CCR8 Antibodies

To determine the binding affinity, the purified chimeric anti-CCR8 antibodies were serially diluted and incubated with 293T-huCCR8 or 293T-cynoCCR8 cells for 30 minutes at 4° C. After washing twice with FACS buffer, diluted Alexa Fluor 647 goat anti-human IgG secondary antibody was added and incubated with 293T-huCCR8 or 293T-cynoCCR8 cells for 30 minutes at 4° C. in the dark. After washing twice with FACS buffer, cells were resuspended with FACS buffer and acquired on a Beckton Dickinson LSR Fortessa™. Titration curves were generated using sigmoid dose-response of nonlinear fit from GraphPad, and the EC50s of representative antibodies are shown in Table 2. As shown in Table 2, all selected clones displayed high affinity against both 293T-huCCR8 and 293T-cynoCCR8 cells, with EC50s of about or lower than 10 nM.

TABLE 2
EC50 of anti-CCR8 antibodies against
293T-huCCR8 and 293T-cynoCCR8 cells

| | EC50 against | EC50 against |
|---|---|---|
| Antibody | 293T-huCCR8 (nM) | 293T-cynoCCR8 (nM) |
| Ch210 | 2.73 | 2.79 |
| Ch211 | 1.75 | 1.57 |
| Ch216-4 | 1.73 | 1.77 |
| Ch224 | 1.00 | 0.85 |
| Ch230-2 | 1.71 | 1.72 |
| Ch231 | 1.80 | 1.90 |
| Ch233 | 0.91 | 0.71 |
| Ch305 | 3.67 | 4.10 |
| Ch327 | 4.32 | 4.76 |
| Ch342-2 | 9.03 | 12.09 |
| Ch357 | 7.12 | 9.96 |
| Ch366-2 | 11.18 | 10.93 |
| Ch404 | 9.08 | 8.19 |
| Ch407 | 2.09 | 2.24 |
| Ch127 | 4.18 | 4.44 |
| Ch326 | 1.36 | 1.39 |

To determine the non-specific binding, the purified chimeric anti-CCR8 antibodies were diluted to 50 nM and incubated with 293T-huCCR1, 293T-huCCR4 and 293T-parental cells for 30 minutes at 4° C. After washing twice with FACS buffer, diluted Alexa Fluor 647™ goat anti-human IgG secondary antibody was added and incubated with 293T-huCCR1, 293T-huCCR4 and 293T-parental cells for 30 minutes at 4° C. in the dark. After washing twice with FACS buffer, cells were resuspended with FACS buffer and acquired on BD LSR Fortessa™ Cell Analyzer. Only very few clones showed non-specific binding against 293T-huCCR1, 293T-huCCR4 or 293T-parental cells and were excluded for further characterization, and only antibodies with specific binding to human and monkey CCR8 were subsequently characterized.

### Example 4. Blocking and Cell Binding of Purified Chimeric Anti-CCR8 Antibodies

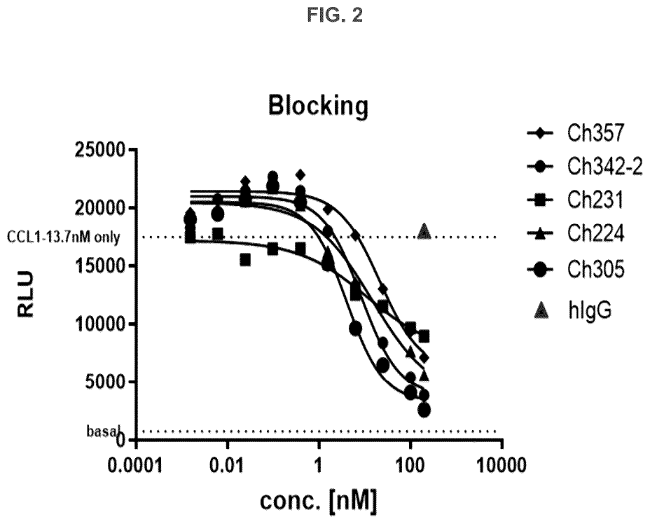

Anti-CCR8 antibody blocks the interaction between the CCL1 ligand and CCR8 receptor and inhibits downstream signaling. DiscoverX Bioassay™ (Eurofins) was used as a cell-based assay to identify the blocking activity of the generated anti-CCR8 antibodies. Briefly, human CCR8 expressed on CHO cells of PathHunter β-Arrestin eXpress GPCR Assay Kit™ (Eurofins) were thawed and seeded to one 96-well assay plate and incubated at 37° C. in 5% CO2. After 48 hours, a series of dilutions of anti-CCR8 antibodies were added to each well and incubated at 37° C. for 30 minutes. 13.7 nM human CCL1 was added to each well and incubated at 37° C. for 90 minutes. Then, Working Detection Solution was added to the assay plate and incubated at room temperature for 1 hour in the dark. The plate was read on a luminescence plate reader. IC50 values were determined by fitting the dose-response data to the four-parameter logistic model with GraphPad Prism. As shown in Table 3 and FIG. 2, the CCR8 chimeric antibody Ch305 showed the strongest ligand blocking activity among all shown variants.

TABLE 3
Comparison of ligand blocking activity of anti-CCR8 antibody

| Antibody | IC50 (nM) | Imax (%) |
|---|---|---|
| Ch357 | 29.97 | 63.56 |
| Ch342-2 | 13.24 | 82.31 |
| Ch231 | 17.84 | 53.04 |
| Ch224 | 24.79 | 72.35 |
| Ch305 | 5.90 | 89.44 |
| hIgG | >200 | 1.29 |

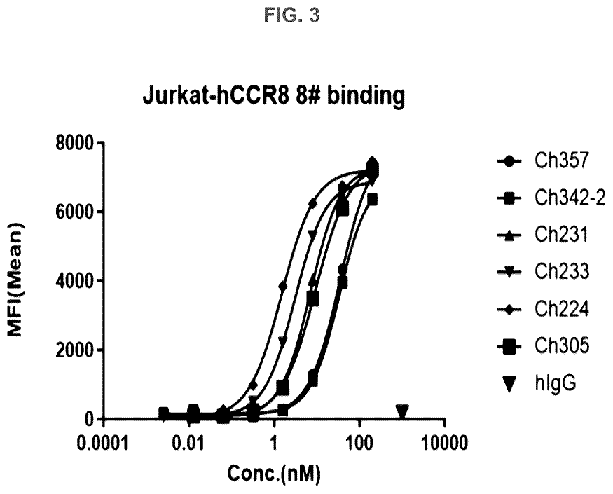

To evaluate the binding activity of anti-CCR8 antibodies to bind CCR8 expressed on living cells, Jurkat cells were engineered to over-express human CCR8. Jurkat-human CCR8 cells were seeded in 96-well plate and incubated with a series of dilutions of anti-CCR8 antibodies. Goat anti-human IgG was used as secondary antibody to detect antibody binding to the cell surface. EC50 values for dose-dependent binding to human CCR8 were determined by fitting the dose-response data to the four-parameter logistic model with GraphPad Prism. As shown in Table 4 and FIG. 3 the CCR8 antibodies had high binding affinity to human CCR8.

TABLE 4
Comparison of binding affinity of anti-CCR8 antibody

| Antibody | EC50 (nM) | Emax (RFU) |
|---|---|---|
| Ch357 | 35.2 | 7061 |
| Ch342-2 | 33.56 | 6371 |
| Ch231 | 7.17 | 7306 |
| Ch233 | 3.06 | 6891 |
| Ch224 | 1.51 | 7469 |
| Ch305 | 9.37 | 7224 |
| hIgG | N/A | 204 |

### Example 5. The Humanization of the Anti-Human CCR8 mAb Ch305

For humanization of the Ch305, human germline IgG genes were searched for sequences that share high degrees of homology to the cDNA sequences of Ch305 variable regions by blasting the human immunoglobulin gene database in IMGT (http://www.imgt.org/IMGT_vquest/share/textes/index.html) and NCBI (http://www.ncbi.nlm.nih.gov/igblast/) websites. The human IGVH and IGVK genes that are present in human antibody repertoires with high frequencies (Glanville et al., Proc Natl Acad Sci USA. 2009 Dec. 1; 106(48):20216-21) and are highly homologous to Ch305 were selected as the templates for humanization.

Humanization was carried out by CDR-grafting (Methods in Molecular Biology, Vol 248: Antibody Engineering, Methods and Protocols, Humana Press) and the humanization antibodies (hu305s) were engineered into the human IgG1 format using an in-house developed expression vector. In the initial round of humanization, mutations from murine to human amino acid residues in framework regions were guided by the simulated 3D structure, and the murine framework residues of structural importance for maintaining the canonical structures of CDRs were retained in the 1st version of humanized 305. Specifically, CDRs of Ch305 VK (SEQ ID NO: 7-9) were grafted into the framework of human germline variable gene IGVK4-1 with several murine framework residues retained. H-CDRs (SEQ ID NO: 4-6) of Ch305 Vh were grafted into the framework of human germline variable gene IGVH4-59 with several murine framework residues retained. In the subsequent humanized variants of Ch305 (Humanized 305 antibodies, or humanized 305s), only the N-terminal half of Kabat HCDR2 was kept, as only the N-terminal half was important for antigen binding according to simulated 3D structure and mutational analysis.

Humanized antibodies were constructed as human full-length antibody format using in-house developed expression vectors that contain constant regions of a human IgG1 and kappa chain, respectively, with easy adapting sub-cloning sites. Expression and preparation of humanized antibodies derived from the clone 305 was achieved by co-transfection of the heavy chain and corresponding light chain constructs into ExpiCHO cells and by purification using a protein A column. The purified antibodies were concentrated to 0.5-5 mg/mL in PBS and stored in aliquots in −80° C. freezer.

Based on the first round humanized 305 template, humanized 305s were further engineered by introducing mutations in CDRs and framework regions to improve binding affinities to human CCR8, humanness, and biophysical properties for therapeutic use in human.

Taken together, the engineered versions of clone 305 resulted in humanized monoclonal antibodies, hu305-4F-2m (SEQ ID NO: 6-9, and 14-19), hu305-4F-21 (SEQ ID NO: 6-9, 14-16, 18, and 20-21), hu305-5W-3a (SEQ ID NO: 6-9, 14-15, and 22-25) and hu305-5P-3a (SEQ ID NO: 4, 6-9, 15, 23, and 25-27) and these were characterized for binding and functional activities. The humanized antibodies hu305-4F-2m, hu305-4F-21, hu305-5W-3a, and hu305-5P-3a can also be referred in shorthand as 4F-2m, 4F-21, 5W-3a, and 5P-3a.

### Example 6. Generation of Afucosylated Anti-CCR8 Antibodies

Removal of core fucose from N-glycans attached to human IgG1 significantly enhances the antibody dependent cell cytotoxicity (ADCC) response (Shields, et al., (2002) J Biol Chem 277, 26733-26740); Shinkawa et al., (2003) J Biol Chem 278, 3466-3473). There are many approaches to reduce core fucosylation. The work presented here utilized the approach of a fucosyltransferase (FUT) inhibitor, 2F-peracetyl-fucose.

186 afucosylated (-AF) CCR8 variants, including Ch305-AF, hu305-4F-21-AF, hu305-4F-2m-AF, hu305-5W-3a-AF, and hu305-5P-3a-AF, were produced by transient transfection of heavy chain and kappa chain containing plasmids in ExpiCHO-s cells. The transfected cells were cultured in shaker flasks using MAX Titer protocol of ExpiCHO expression system. 2F-peracetyl-fucose concentration of 100 μM was added to the culture. Cell supernatant was harvested on day 14 and filtered through a 0.2 μm filter for further analysis.

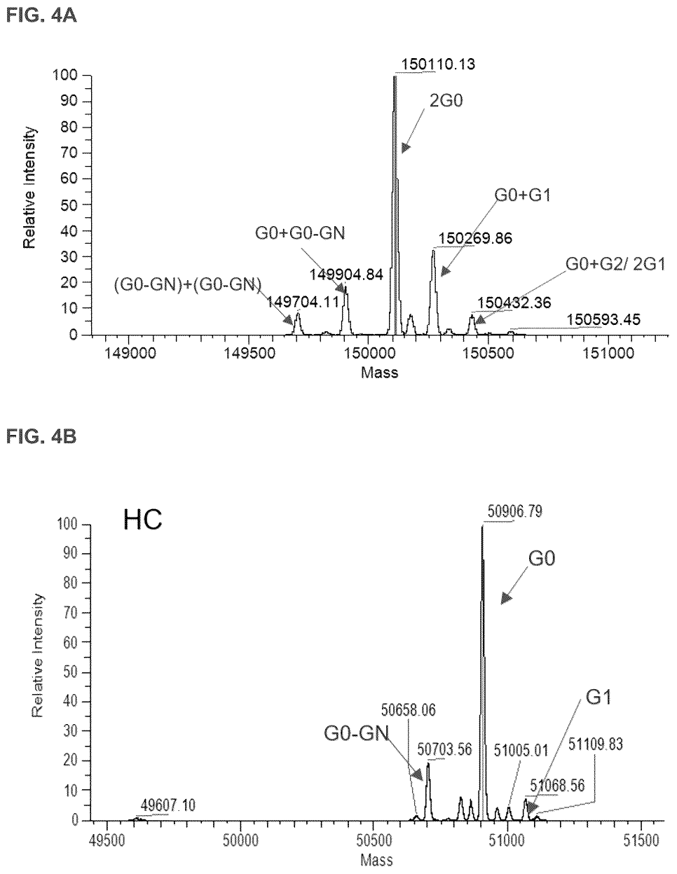

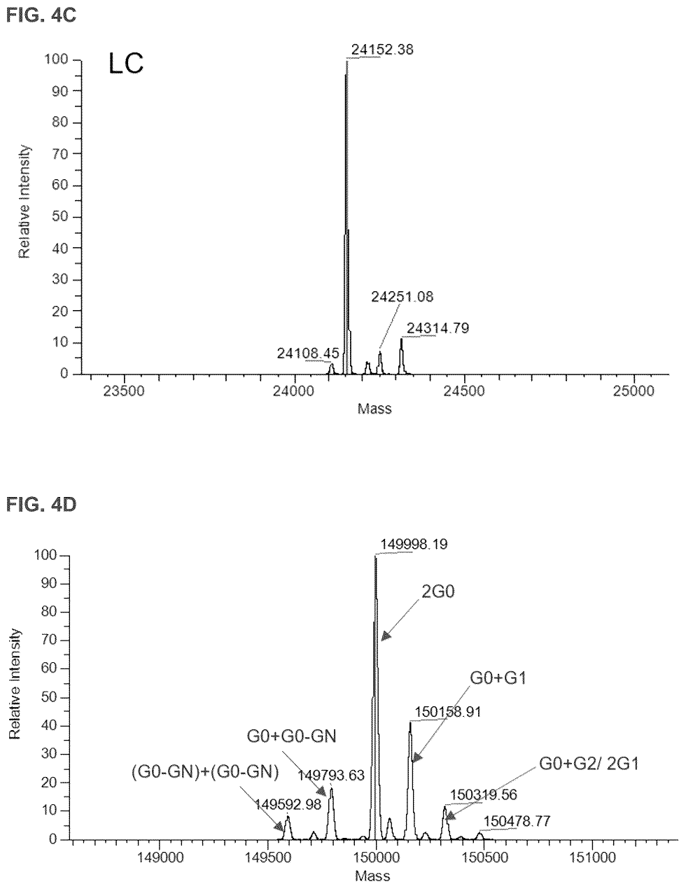

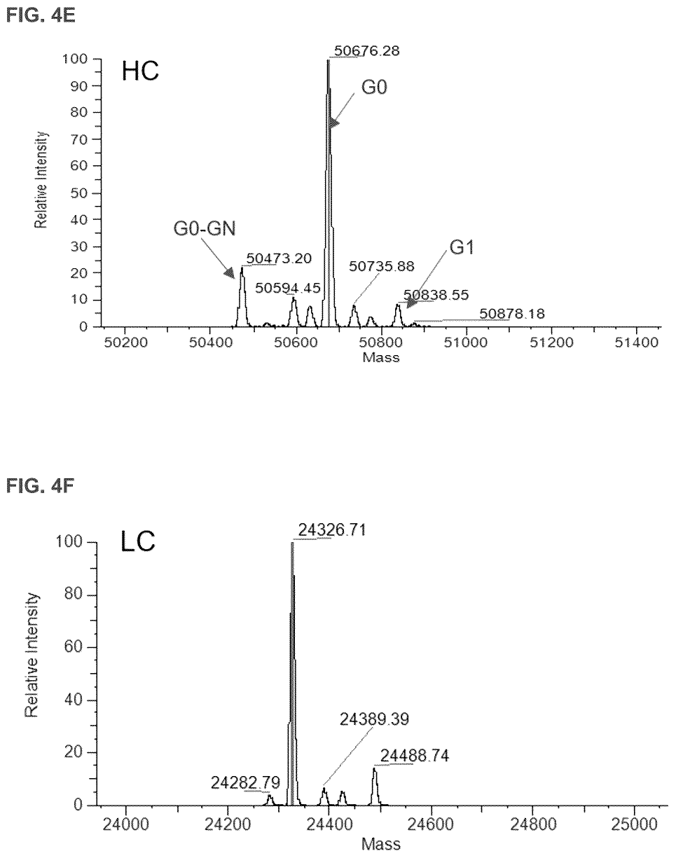

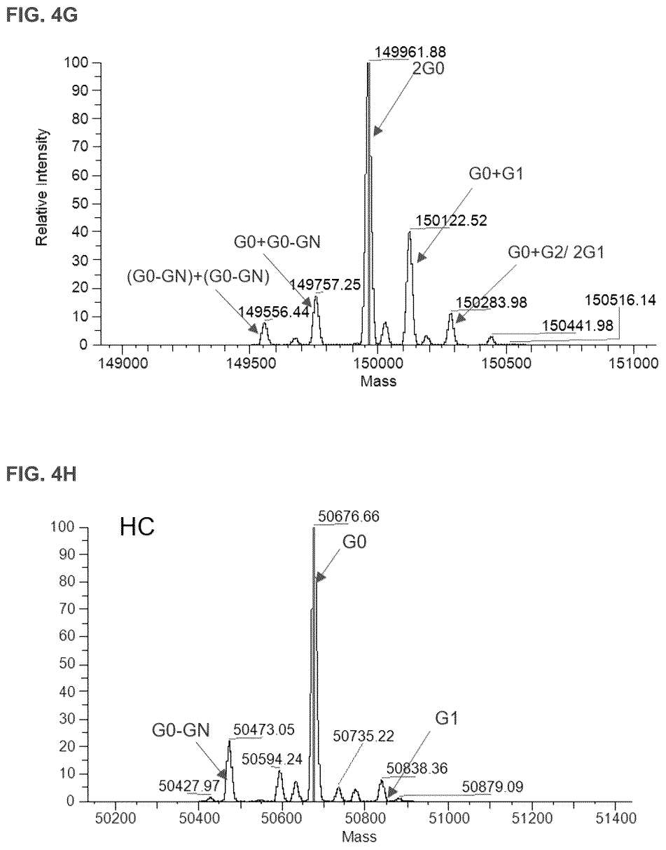

All these antibodies purified with a Protein A chromatography capture step and/or other polishing purification steps under platform conditions. Glycan profiles of Ch305-AF, hu305-4F-21-AF and hu305-4F-2m-AF were analyzed by Intact and reduced Mass Spectrometry, the results of which are shown in Table 5-10 and FIG. 4A-I. Glycan analysis showed that no fucose related N-glycosylated mass was detected at 100 μM 2F-peracetyl-fucose.

TABLE 5
Intact mass results of Ch305-AF

| | | | Theoretical | Measured | |
|---|---|---|---|---|---|
| | | | mass | mass | ΔMass |
| Samples | Assay | Component | (Da) | (Da) | (Da) |
| Ch305- | Intact | IM: −2K, 2pE(Q), | 149703.15 | 149704.11 | −0.96 |
| AF | mass | (G0 − GN) + | | | |
| | | (G0 − GN) | | | |
| | | IM: −2K, 2pE(Q), | 149906.15 | 149904.84 | 1.31 |
| | | G0 + (G0 − GN) | | | |
| | | IM: −2K, 2pE(Q), | 150109.15 | 150110.13 | −0.98 |
| | | 2G0 | | | |
| | | IM: −2K, 2pE(Q), | 150271.15 | 150269.86 | 1.29 |
| | | G0 + G1 | | | |
| | | IM: −2K, 2pE(Q), | 150433.15 | 150432.36 | 0.79 |
| | | 2G1/G0 + G2 | | | |

TABLE 6
Reduced mass results of Ch305-AF

| | | | Theoretical | Measured | |
|---|---|---|---|---|---|
| | | | mass | mass | ΔMass |
| Samples | Assay | Component | (Da) | (Da) | (Da) |
| Ch305- | Reduced | HC: −K, pE(Q), | 50702.82 | 50703.56 | −0.74 |
| AF | mass | G0 − GN | | | |
| | | HC: −K, pE(Q), G0 | 50905.82 | 50906.79 | −0.97 |
| | | HC: −K, pE(Q), G1 | 51067.82 | 51068.56 | −0.74 |
| | | LC | 24152.75 | 24152.38 | 0.37 |

TABLE 7
Intact mass results of hu305-4F-21-AF

| Samples | | Theoretical | Measured | ΔMass |
|---|---|---|---|---|
| Assay | Component | mass(Da) | mass(Da) | (Da) |
| Intact | IM: −2K, 2pE(Q), | 149591.02 | 149592.98 | −1.96 |
| 4F-2l-AF | (G0 − GN) + | | | |
| mass | (G0 − GN) | | | |
| | IM: −2K, 2pE(Q), | 149794.02 | 149793.63 | 0.39 |
| | G0 + (G0 − GN) | | | |
| | IM: −2K, 2pE(Q), | 149997.02 | 149998.19 | −1.17 |
| | 2G0 | | | |
| | IM: −2K, 2pE(Q), | 150159.02 | 150158.91 | 0.11 |
| | G0 + G1 | | | |
| | IM: −2K, 2pE(Q), | 150321.02 | 150319.56 | 1.46 |
| | 2G1/G0 + G2 | | | |

TABLE 8
Reduced mass results of hu305-4F-21-AF

| | | | Theoretical | Measured | ΔMass |
|---|---|---|---|---|---|
| Samples | Assay | Component | mass(Da) | mass(Da) | (Da) |
| 4F-2l-AF | Reduced | HC: −K, pE(Q), | 50472.68 | 50473.2 | −0.52 |
| | mass | G0 − GN | | | |
| | | HC: −K, pE(Q), G0 | 50675.68 | 50676.28 | −0.6 |
| | | HC: −K, pE(Q), G1 | 50837.68 | 50838.55 | −0.87 |
| | | LC | 24326.82 | 24326.71 | 0.11 |

TABLE 9
Intact mass results of hu305-4F-2m-AF

| | | | Theoretical | Measured | ΔMass |
|---|---|---|---|---|---|
| Samples | Assay | Component | mass(Da) | mass(Da) | (Da) |
| 4F-2m-AF | Intact | IM: −2K, 2pE(Q), | 149554.94 | 149556.44 | −1.5 |
| | mass | (G0 − GN) + | | | |
| | | (G0 − GN) | | | |
| | | IM: −2K, 2pE(Q), | 149757.94 | 149757.25 | 0.69 |
| | | G0 + (G − GN) | | | |
| | | IM: −2K, | 149960.94 | 149961.88 | −0.94 |
| | | 2pE(Q), 2G0 | | | |
| | | IM: −2K, 2pE(Q), | 150122.94 | 150122.52 | 0.42 |
| | | G0 + G1 | | | |
| | | IM: −2K, 2pE(Q), | 150284.94 | 150283.98 | 0.96 |
| | | 2G1/G0 + G2 | | | |

TABLE 10
Reduced mass results of hu305-4F-2m-AF

| | | | Theoretical | Measured | ΔMass |
|---|---|---|---|---|---|
| Samples | Assay | Component | mass(Da) | mass(Da) | (Da) |
| 4F-2m-AF | Reduced | HC: −K, pE(Q), | 50472.68 | 50473.05 | −0.37 |
| | mass | G0 − GN | | | |
| | | HC: −K, pE(Q), G0 | 50675.68 | 50676.66 | −0.98 |
| | | HC: −K, pE(Q), G1 | 50837.68 | 50838.36 | −0.68 |
| | | LC | 24308.78 | 24308.61 | 0.17 |

### Example 7. Binding Activities of Humanized Antibodies to Human CCR8

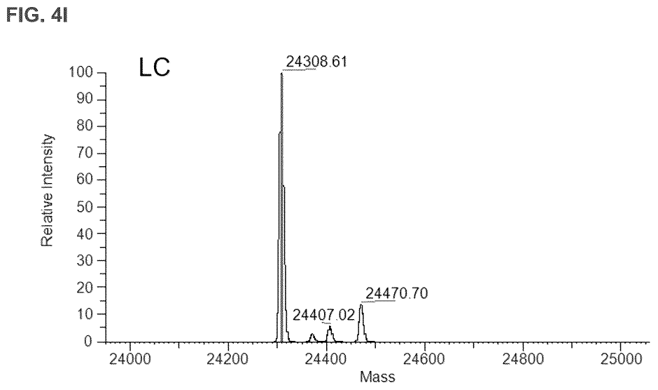

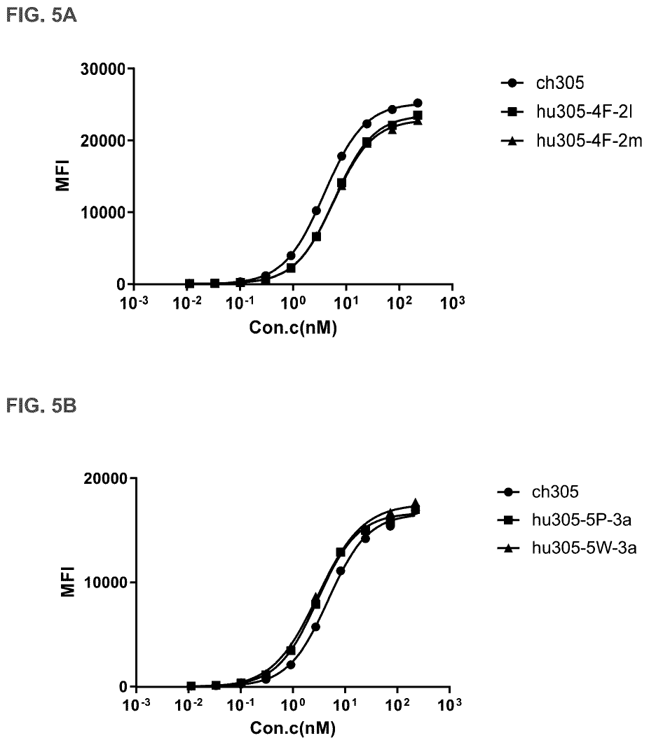

To evaluate the binding activities of anti-CCR8 antibodies to bind native CCR8 on live cells, Jurkat cells (ATCC TIB-15) were engineered to over-express human CCR8. Live Jurkat/huCCR8 cells were seeded in 96-well plate, and were incubated with a series of dilutions of anti-CCR8 antibodies. Alexa Fluor® 647 anti-Human IgG Fc Antibody (Biolegend cat: 410714) was used as a secondary antibody to detect antibody binding to CCR8 expressed on the cell surface. EC50 values for dose-dependent binding to human native CCR8 were determined by fitting the dose-response data to the four-parameter logistic model with GraphPad Prism. As shown in FIG. 5 and Table 11. The humanized antibodies retained binding affinity to native human CCR8 indicating that humanization resulted in little to no loss of ligand blocking activity when compared to the original clone.

TABLE 11
Jurkat/huCCR8 FACS binding affinities and
Emax of different versions of 305

| | Test1 | | Test2 | |
|---|---|---|---|---|
| Antibody | Ec50 (nM) | Emax (MFI) | Ec50 (nM) | Emax (MFI) |
| Ch305-AF | 3.88 | 25245 | 4.75 | 16608 |
| hu305-4F-2l-AF | 6.01 | 23549 | — | — |
| hu305-4F-2m-AF | 5.83 | 22884 | — | — |
| hu305-5W-3a-AF | — | — | 2.98 | 16689 |
| hu305-5P-3a-AF | — | — | 2.99 | 17533 |

The “—” in Table 11 means not tested.

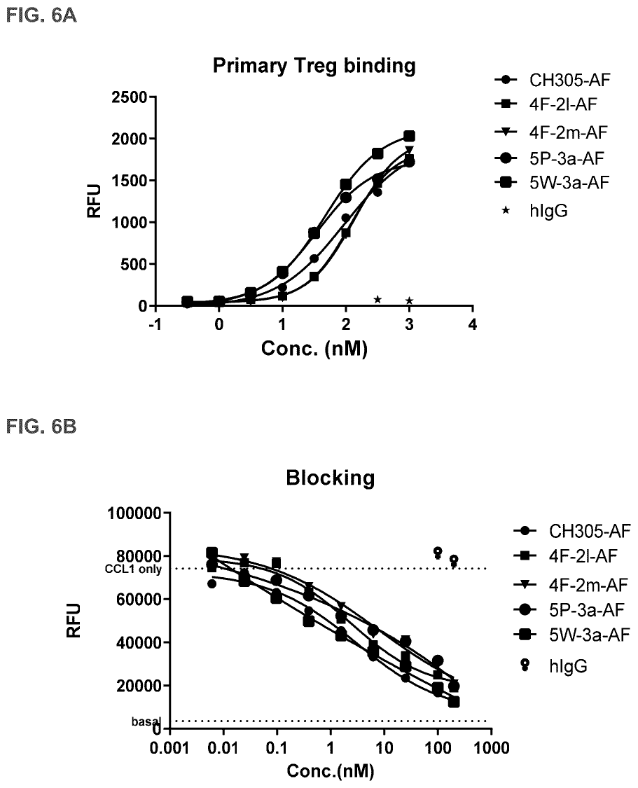

Treg binding of the humanized antibodies was measured on Treg cells from human PBMC. Briefly, CD25+ cells were isolated from human PBMC. The cells were stimulated with IL-2, TGFβ and anti-CD3/CD28 beads for high CCR8-expression level Treg cells. Next, Treg cells were seeded in 96-well plate and incubated with a series of dilutions of anti-CCR8 antibodies. Goat anti-human IgG was used as secondary antibody to detect antibody binding to the cell surface. EC50 values for dose-dependent binding to human CCR8 were determined by fitting the dose-response data to the four-parameter logistic model with GraphPad Prism. As shown in FIG. 6A and Table 12 the hu305-5W-3a had the highest binding affinity to CCR8 on Treg cells of the humanized antibodies. As noted in Table 12 and FIG. 6A below, the hu305-5W-3a antibody also had the lowest IC50 regarding reducing ligand-receptor interaction.

TABLE 12
Comparison of Primary Treg binding
affinity of humanized 305 antibodies

| Antibody | EC50(nM) | Emax(RLU) |
|---|---|---|
| Ch305-AF | 90.7 | 1748 |
| hu305-4F-2l-AF | 116.6 | 1761 |
| hu305-4F-2m-AF | 135.9 | 1860 |
| hu305-5P-3a-AF | 35.2 | 1722 |
| hu305-5W-3a-AF | 47.6 | 2031 |
| hIgG | NA | 60 |

CCR8 blocking activities of humanized antibodies were also determined and shown in FIG. 6B and Table 13.

TABLE 13
Comparison of blocking activity of humanized antibodies

| Antibody | IC50(nM) | Imax(%) |
|---|---|---|
| Ch305-AF | 1.84 | 87.8 |
| hu305-4F-2l-AF | 6.19 | 78.0 |
| hu305-4F-2m-AF | 13.05 | 75.6 |
| hu305-5P-3a-AF | 10.53 | 77.1 |
| hu305-5W-3a-AF | 1.82 | 87.3 |
| hIgG | NA | NA |

### Example 8. Determining the Cross Reactivity of Anti-CCR8 Antibodies

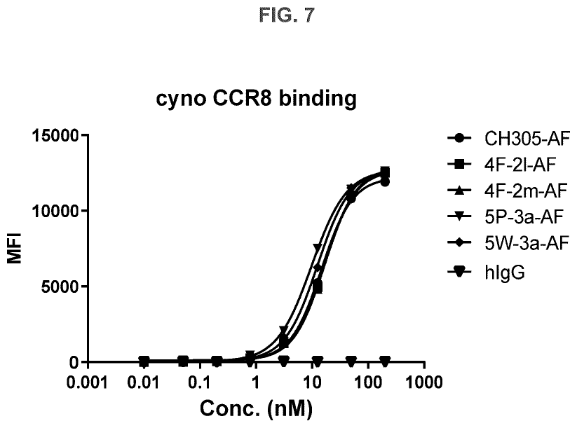

To evaluate the cross reactivity of humanized antibody to cynomolgus (cyno) monkey CCR8, cynoCCR8 was overexpressed in 293T. 293T-cynoCCR8 cells were seeded in 96-well plate and incubated with a series of dilutions of anti-CCR8 antibodies. Goat anti-human IgG was used as secondary antibody to detect antibody binding to the cell surface. EC50 values for dose-dependent binding to cyno CCR8 were determined by fitting the dose-response data to the four-parameter logistic model with GraphPad Prism. As shown in FIG. 7 and Table 14, the humanized CCR8 antibodies exhibit good binding affinity to cyno CCR8.

TABLE 14
Comparison of cross reactivity of humanized Ch305 antibodies

| Antibody | EC50(nM) | Emax (RLU) |
|---|---|---|
| Ch305-AF | 14.79 | 11900 |
| hu305-4F-2l-AF | 16.74 | 12500 |
| hu305-4F-2m-AF | 16.62 | 12300 |
| hu305-5P-3a-AF | 9.83 | 12600 |
| hu305-5W-3a-AF | 12.52 | 12400 |
| IgG | NA | 9.75 |

### Example 9. CCR8 Antibodies Enhanced NK Cell Mediated ADCC

Anti-CCR8 antibodies induced ADCC was determined by measuring Treg cell depletion by NK cell upon antibody treatment. Briefly, NK92-MI-CD16a-F158 cell line was generated as effector cells. The CCR8 expressed-Treg was used as target cells. Equal numbers (5×10⁴) of target cells and effector cells were added to 96-well plates with a series of dilutions of anti-CCR8 antibodies and co-cultured for 24 hours at 3° C. Cytotoxicity was evaluated by flow cytometry. The results were calculated by the formula shown below:

$$\% Cytotoxicity = \frac{\begin{matrix}CD 25 + \% of no antibody well - \\ CD 25 + \% of CCR 8 antibody well\end{matrix}}{CD 25 \% of no antibody well} \times 100$$

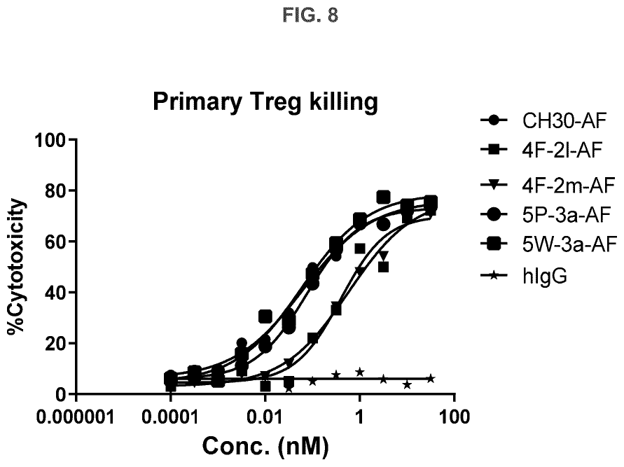

As shown in FIG. 8 and Table 15, all of the anti-CCR8 antibodies tested had ADCC activity with hu305-5W-3a demonstrating the highest potency in killing CCR8 expressing targets via ADCC in a dose dependent manner.

TABLE 15
Comparison of cytotoxicity of humanized antibodies

| Antibody | EC50 (nM) | Emax (%) |
|---|---|---|
| Ch305-AF | 0.064 | 75.1 |
| hu305-4F-2l-AF | 0.396 | 72.3 |
| hu305-4F-2m-AF | 0.585 | 70.9 |
| hu305-5P-3a-AF | 0.077 | 73.7 |
| hu305-5W-3a-AF | 0.059 | 75.5 |
| hIgG | NA | 8.6 |

### Example 10. Epitope Analysis of Anti-CCR8 Antibodies

To analyze the epitopes of anti-CCR8 antibodies, Jurkat cells overexpressing human CCR8 (Jurkat-huCCR8) were incubated with 200 nM of unlabeled anti-CCR8 or human IgG1 isotype control antibodies (first antibody) for 30 minutes at 4° C. Without washing, 200 nM of APC labeled anti-CCR8 or human IgG1 isotype control antibodies (secondary antibody) were added and incubated for another 30 minutes at 4° C. After washing twice with FACS buffer, cells were resuspended with FACS buffer and acquired on BD LSR Fortessa™ Cell Analyzer. The readouts were normalized as the % of the maximum binding of the individual APC labeled antibody compared with unlabeled human IgG1 isotype control antibody as first antibody. The epitope binning results are summarized in Table 16. Low value from table cell indicates it's antibody pair sharing similar epitope. As shown in Table 16, Ch211 and Ch305 are in the same epitope bin, which have similar epitope; Ch366-2, reference anti-CCR8 antibodies 4A19 (as disclosed in WO2021194942A1) and 433H (commercially available from BD Biosciences) are in the same epitope bin; and Ch127 and Ch326 are in another two different epitope bins.

TABLE 16
Epitope binning by FACS

| | 2nd Ab (APC labeled) | | | | | | | |
|---|---|---|---|---|---|---|---|---|
| | | Ch211 | Ch305 | Ch127 | Ch326 | Ch366-2 | 4A19 | 433H |
| 1st Ab | Ch211 | 6.38 | 5.13 | 78.57 | 46.95 | 57.59 | 89.79 | 78.77 |
| (unlabeled) | Ch305 | 8.99 | 7.20 | 67.41 | 45.52 | 48.28 | 93.33 | 81.98 |
| | Ch127 | 87.97 | 56.72 | 33.66 | 7.43 | 43.69 | 83.81 | 58.06 |
| | Ch326 | 88.77 | 63.78 | 82.50 | 42.57 | 81.88 | 96.04 | 86.34 |
| | Ch366-2 | 58.04 | 24.22 | 6.31 | 1.60 | 6.38 | 14.41 | 8.94 |
| | 4A19 | 79.85 | 67.67 | 3.55 | 2.02 | 13.63 | 4.55 | 2.46 |
| | 433H | 39.85 | 28.94 | 2.55 | 1.87 | 14.25 | 8.43 | 3.03 |

To determine whether anti-CCR8 antibodies bind to the N-terminal domain of human CCR8, ELISA plates were coated with human CCR8 N-terminal domain-mouse Fc tag fusion protein (SEQ ID NO: 28 and 29) overnight at 4° C. After washing and blocking, serially diluted anti-CCR8 antibodies were added to plates and incubated for 1 hour at room temperature. After washing, diluted HRP goat anti-human IgG Fc or HRP goat anti-mouse kappa chain (for 433H) secondary antibodies were added to plates and incubated for 45 minutes at room temperature. After washing, TMB substrate was added to plates for developing and then stopped by adding TMB substrate stop solution. The absorbance was read at 450 nm using a plate reader. Titration curves were generated using sigmoid dose-response of nonlinear fit from GraphPad, and the EC50s are shown in Table 17. As shown in Table 17, Ch127, Ch326, Ch366-2, 4A19 and 433H displayed high binding affinity against human CCR8 N-terminal protein with EC50s<1 nM. In contrast, CH305 showed low binding affinity against human CCR8 N-terminal protein with an EC50 of 5 nM. CH211 only showed very weak binding activity against human CCR8 N-terminal protein at highest concentration of 640 nM, indicating they are binding to CCR8 N-terminal loop. In contrast, Ch305 showed low binding affinity against human CCR8 N-terminal protein with an EC50 of 5 nM, which may be partial CCR8 N-terminal loop binder. Ch211 only showed very weak binding activity against human CCR8 N-terminal protein at highest concentration of 640 nM. It is a non CCR8 N-terminal loop binder.

TABLE 17
EC50 of anti-CCR8 antibodies against
human CCR8 N-terminal protein

| Antibody | EC50 (nM) |
|---|---|
| Ch211 | >640 |
| Ch305 | 5.01 |
| Ch127 | 0.10 |
| Ch326 | 0.13 |
| Ch366-2 | 0.14 |
| 4A19 | 0.11 |
| 433H | 0.94 |

### Example 11. Epitope Mapping of Antibody Anti-CCR8 Antibodies

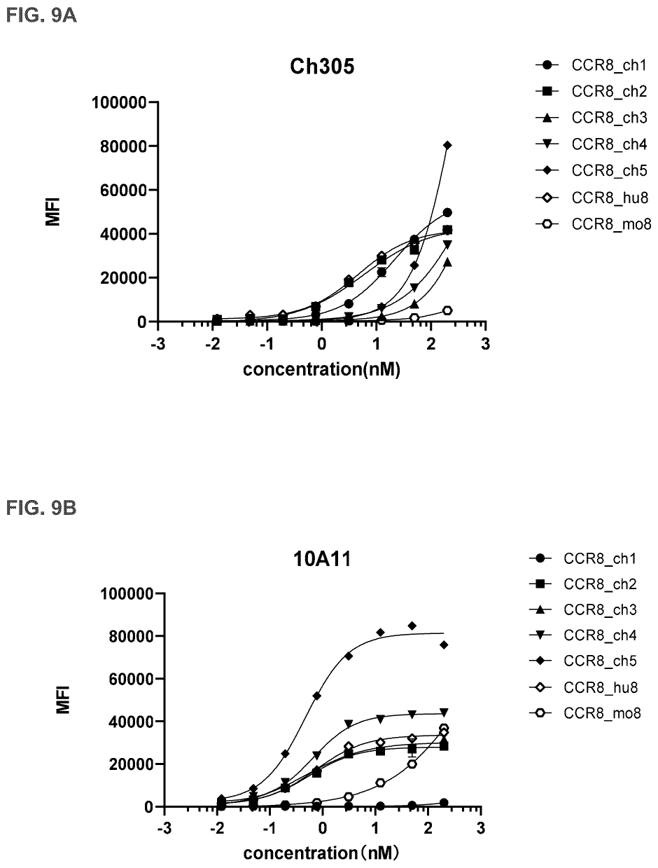

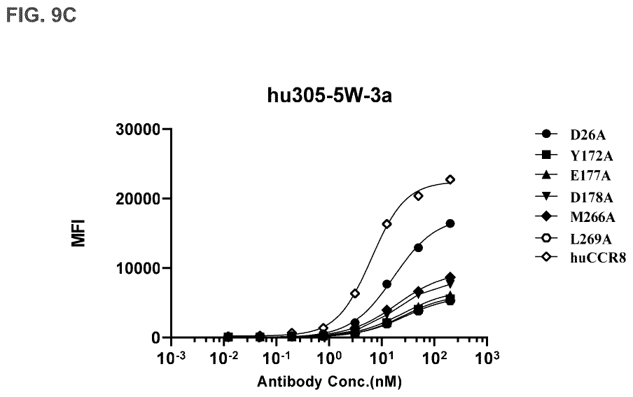

To study the binding epitope of anti-CCR8 mAb Ch305, domain swapped CCR8 was generated by grafting amino acids from extracellular regions of mouse CCR8 into human CCR8, or vice versa. More specifically, the mapping strategy utilizes of the fact that Ch305 can bind human CCR8 (SEQ ID NO:1), but not mouse CCR8 (SEQ ID NO:3) (FIG. 9A). The alignment of the amino acid sequences of human CCR8 (huCCR8) and mouse CCR8 (muCCR8) is shown in FIG. 1A. The amino acid residues of the extra cellular domains of CCR8 are indicated by underlining. The extracellular domains (ECD) of both mouse and human CCR8 proteins are indicated and labeled “N-terminal”, “ECL1”, “ECL2” and “ECL3” above the sequence of each region on FIG. 1A. The gene encoding the full length of human CCR8 (SEQ ID NO: 1, UniProtKB: P51685) and mouse CCR8 (SEQ ID NO: 3, UniProtKB: P56484) were cloned into pcDNA 3.4 vectors. The chimeric constructs were generated in which the extracellular domains of human CCR8 including the N-terminal region (amino acids 1-35 of SEQ ID NO: 1), the ECL1 region (amino acids 94-107 of SEQ ID NO: 1), the ECL2 region (amino acids 172-202 of SEQ ID NO: 1) and the ECL3 region (amino acids 264-280 of SEQ ID NO: 1) are substituted with the corresponding regions of mouse CCR8, the N-terminal region (amino acids 1-33 of SEQ ID NO: 3), ECL1 region (amino acids 92-105 of SEQ ID NO: 3), ECL2 region (amino acids 170-200 of SEQ ID NO: 3) and ECL3 region (amino acids 262-278 of SEQ ID NO: 3), respectively. Based on this swapping strategy, the absence or reduction of anti-CCR8 antibodies to the specific construct would mean that specific domain (e.g., ECL1) is at least partially required for anti-CCR8 antibody binding.

Table 18 show in a tabular form which mouse (murine) regions were swapped into human CCR8 (human).

TABLE 18
Chimeric CCR8 constructs with domain swapping

| Chimeric constructs | N-terminal | ECL1 | ECL2 | ECL3 |
|---|---|---|---|---|
| CCR8_Ch1 | murine | human | Human | human |
| CCR8_Ch2 | human | murine | human | human |
| CCR8_Ch3 | human | human | murine | human |
| CCR8_Ch4 | human | human | human | murine |
| CCR8_Ch5 | human | murine | murine | murine |

The plasmids containing these chimeric CCR8 constructs, and human CCR8 (CCR8-hu8) and mouse CCR8 (CCR8-mo8) as control, were used to transfect Expi293™ cells for transient protein expression, after which cells were incubated with a serial dilution of the purified Ch305 as well as a reference antibody 10A11 from Shionogi (US 2022/0064312 A1). The binding of each construct was evaluated detection with Alexa Fluor 647 Rabbit Anti-Human IgG (Cat.: 309-605-008 Jackson ImmunoResearch). The data in FIG. 9A shows that Ch305 specifically binds to human CCR8 but not mouse CCR8. The binding was significantly reduced in CCR8 constructs containing murine ECL2 or ECL3 substituted regions, indicating the key roles of these regions for anti-CCR8 antibody binding. In addition, replacing the human CCR8 N-terminal region with mouse CCR8 N-terminal (CCR8_Ch1) also weakly impairs the affinity, which means human CCR8 N-terminal contributes to the binding activity of human CCR8 with Ch305. In the meanwhile, substituting mouse CCR8 N-terminal with human CCR8 N-terminal (CCR8_Ch5) rescues the binding activity of murine CCR8 with Ch305 to some extent much weaker than the binding activity of wild type human CCR8 to Ch305, which means human CCR8 N-terminal contributes but weakly or partly to the binding activity of human CCR8 with Ch305. These data demonstrate that Ch305 binds to N-terminal region, ECL2 and ECL3 of human CCR8, which means the epitope of Ch305 and its humanized antibodies is composed of N-terminal region, ECL2 and ECL3 of human CCR8. In comparison, the reference antibody 10A11 binds to the N-terminal region of CCR8 only and it has no binding to ECL1, ECL2 and ECL3, that is, the epitope of 10A11 on human CCR8 locates only on N-terminal of human CCR8 (FIG. 9B).

To more precisely determine the specific amino acid residues that are crucial for antibody binding to human CCR8, point mutations were introduced to ECL2, ECL3 and N-terminal regions of human CCR8, as shown in the first column of Table 19. To evaluate the impact of each mutant, humanized anti-CCR8 antibody hu305-5W-3a was used to react with transfected Expi293 cells as described above with the concentration of 50 nM, 10 nM and 1 nM (results summarized in Table 19). The critical amino acids for hu305-5W-3a binding are marked with an “X” indicating a loss of >50% compared with wild type huCCR8, which is considered significant.

The preliminary data indicate that the antibody hu305-5W-3a recognizes amino acid D26 on the N-terminal domain, Y172, E177, D178 on ECL2 and M266, L269 from ECL3 (numbering according to SEQ ID NO: 1). Mutation on these sites will significantly reduce the binding of hu305-5W-3a. In comparison, the 10A11 reference antibody recognizes D14, Y17, D19 on the N-terminal domain of huCCR8 as the mutation on these sites totally abolished the binding, indicating no overlap in epitope between the reference antibody 10A11 and hu305-5W-3a. To further confirm the critical roles of these sites for binding, the transfected Expi293 cells were stained with a serial dilution of hu305-5W-3a from 200 nM followed by detection with Alexa Fluor 647 Rabbit Anti-Human IgG (Cat.: 309-605-008 Jackson ImmunoResearch) to obtain a full binding curve. The data in FIG. 9C and Table 20 shows the influence of these key residues for hu305-5W-3a binding to CCR8. Among these sites, Y172, E177 and L269 are considered to be the most critical epitopes, and substitution them with Ala will lead to more than 4 folds decrease of EC50. D178 and M266 are considered as 2^{nd} tier epitopes having media impact on Hu305-5W-3a binding. Substitution them with Ala will lead to ˜3 folds decrease of EC50. Lastly, D26 from the N-terminal domain has least impact compared with other key residues and mutation of which leads to a decrease of EC50 for more than 2 folds. Taken together, these data agree well with the notion that Hu305-5W-3a recognizes a conformational epitope consisted of N-terminal, ECL2 and ECL3.

TABLE 19
Hu305-5W-3a epitope mapping summary

| Mutant | Domain | 5W-3a | 10A11 |
|---|---|---|---|
| huCCR8 | N-terminal | — | — |
| D2A | N-terminal | | |
| Y3A | N-terminal | | |
| T4A | N-terminal | | |
| L5A | N-terminal | | |
| D6A | N-terminal | | |
| L7A | N-terminal | | |
| S8A | N-terminal | | |
| V9A | N-terminal | | |
| T10A | N-terminal | | |
| T11A | N-terminal | | |
| V12A | N-terminal | | |
| T13A | N-terminal | | |
| D14A | N-terminal | | X |
| Y15A | N-terminal | | |
| Y16A | N-terminal | | |
| Y17A | N-terminal | | X |
| P18A | N-terminal | | |
| D19A | N-terminal | | X |
| I20A | N-terminal | | |
| F21A | N-terminal | | |
| S22A | N-terminal | | |
| S23A | N-terminal | | |
| P24A | N-terminal | | |
| C25A | N-terminal | | |
| D26A | N-terminal | X | |
| E28A | N-terminal | | |
| L29A | N-terminal | | |
| I30A | N-terminal | | |
| Q31A | N-terminal | | |
| T32A | N-terminal | | |
| N33A | N-terminal | | |
| G34A | N-terminal | | |
| K35A | N-terminal | | |
| Y172A | ECL2 | X | |
| Q173A | ECL2 | | |
| V174A | ECL2 | | |
| S176A | ECL2 | | |
| E177A | ECL2 | X | |
| D178A | ECL2 | X | |
| G179A | ECL2 | | |
| V180A | ECL2 | | |
| L181A | ECL2 | | |
| Q182A | ECL2 | | |
| C183A | ECL2 | | |
| Y184A | ECL2 | | |
| S185A | ECL2 | | |
| F186A | ECL2 | | |
| Y187A | ECL2 | | |
| N188A | ECL2 | | |
| Q189A | ECL2 | | |
| Q190A | ECL2 | | |
| T191A | ECL2 | | |
| L192A | ECL2 | | |
| K193A | ECL2 | | |
| W194A | ECL2 | | |
| K195A | ECL2 | | |
| I196A | ECL2 | | |
| F197A | ECL2 | | |
| T198A | ECL2 | | |
| N199A | ECL2 | | |
| F200A | ECL2 | | |
| K201A | ECL2 | | |
| M202A | ECL2 | | |
| H264A | ECL3 | | |
| S265A | ECL3 | | |
| M266A | ECL3 | X | |
| H267A | ECL3 | | |
| I268A | ECL3 | | |
| L269A | ECL3 | X | |
| D270A | ECL3 | | |
| G271A | ECL3 | | |
| C272A | ECL3 | | |
| S273A | ECL3 | | |
| I274A | ECL3 | | |
| S275A | ECL3 | | |
| Q276A | ECL3 | | |
| Q277A | ECL3 | | |
| L278A | ECL3 | | |
| T279A | ECL3 | | |
| Y280A | ECL3 | | |

TABLE 20
Hu305-5W-3a binding to each critical epitope mutant

| Mutant | Domain | EC50 of Hu305-5W-3a (nM) |
|---|---|---|
| huCCR8 | — | 6.3 |
| D26A | N-terminal | 16.8 |
| Y172A | ECL2 | 26.3 |
| E177A | ECL2 | 25.4 |
| D178A | ECL2 | 18.3 |
| M266A | ECL3 | 19.4 |
| L269A | ECL3 | 27.4 |

### Example 12. Anti-mCCR8 mAb and Anti-PD-1 mAb Synergistically Inhibit Tumor Growth in CT26 Mouse Colon Cancer Model

The efficacy of anti-murine CCR8 antibody as single agent or in combination with an anti-mPD-1 Ab (Ch15mt) was tested in a CT26 mouse colon cancer model. Ch15mt is a murinized monoclonal antibody against mouse PD-1, which was generated by immunizing rat with the recombinant mouse PD-1 protein, followed by murinizing the rat Fc to mouse IgG1 isotype (Chen X. et al., Front Immunol. 2022 Feb. 22; 13:828319). The Asp265 on the heavy chain was further substituted with Ala to eliminate the Fc receptor binding. Murine CT26 colon cancer cells (3×10⁴) were subcutaneously implanted in BALB/C mice. After tumor cell implantation, tumor length (L) and width (W) will be measured twice weekly using electronic calipers, and the volume will be expressed in mm³ using the formula: V=0.5 (a×b²) where a and b are the long and short diameters of the tumor, respectively. When tumors reached a mean volume of approximately 100 mm³ in size, mice were randomly allocated into 7 groups (15 per group), and injected intraperitoneally with mAbs once a week for three weeks. PBS was administered as vehicle control. Tumor growth inhibition (TGI) was calculated using the following formula: TGI=[1−(treated Tt−treated T0)/(vehicle Tt−vehicle T0)]×100, in which treated Tt=treated tumor volume at Time t, treated T0=treated tumor volume at Time 0, vehicle Tt=vehicle tumor volume at Time t, and vehicle T0=vehicle tumor volume at Time 0.

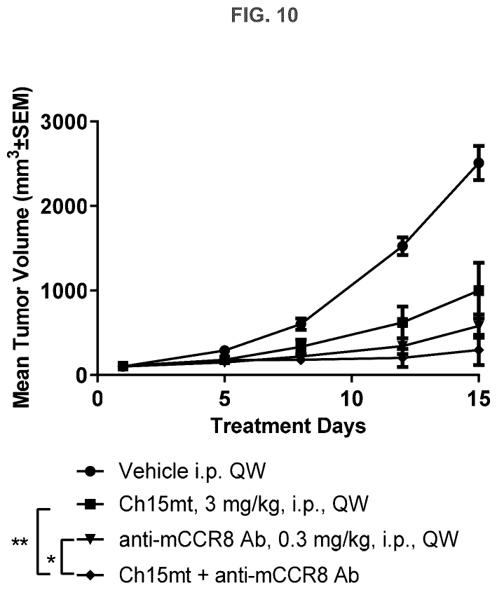

The results demonstrated that single agent anti-murine CCR8 (0.3 mpk) and anti-mPD-1 (3 mpk) treatments induced relatively strong anti-tumor efficacy, with the anti-CCR8 antibody showing higher TGI than the anti-PD-1 antibody as single agent therapy (FIG. 10). In addition, the administration of anti-CCR8 and anti-PD-1 mAb in combination demonstrated a synergistic effect evidenced by the higher TGI level. Combination treatment almost completely induced tumor rejection in 8 out of 16 mice, while the tumor free rate in anti-CCR8 and anti-PD-1 single agent treatment group was only 0 and 2 out of 15 (FIG. 10 and Table 21). This demonstrated that anti-CCR8 treatment can synergistically enhance the anti-tumor activity of anti-PD-1 in a mouse colon tumor model.

TABLE 21
The combination efficacy of anti-murine CCR8 and anti-murine
PD-1 in a murine CT26 colon tumor mouse model

| | Dose | Mean tumor | TGI (%) | Tumor Free |
|---|---|---|---|---|
| Treatment | (mpk) | vol. at day 15 | at day 15 | at day 22 |
| PBS | — | 2520.5 | — | 0 |
| Ch15mt | 3 | 998 | 63 | 2 |
| Anti-CCR8 | 0.3 | 578.3 | 80 | 0 |
| Anti- | 0.3 + | 295 | 92 | 8 |
| CCR8 + | 3 | | | |
| Ch15mt | | | | |

### Example 13. Pharmacodynamic Responses to Anti-CCR8 mAb Treatment in a MC38 Mouse Colon Cancer Model

The pharmacodynamic Treg depletion effect of anti-CCR8 antibody hu305-4F-21-AF and hu305-5W-3a-AF were measured in a MC38 mouse colon cancer model. Murine MC38 colon tumor cells (purchased from Kerafast, ENH204-FP) (1×10⁶) were subcutaneously implanted in C57 mice transgenic for human CCR8 (SMOC, Shanghai China). After tumor cell implantation, tumor length (L) and width (W) will be measured started from Day 6-9 (when tumor volume approximates 100 mm³) at a 2 or 3-day interval using electronic calipers, and the volume will be expressed in mm³ using the formula: V=0.5(a×b²) where a and b are the long and short diameters of the tumor.

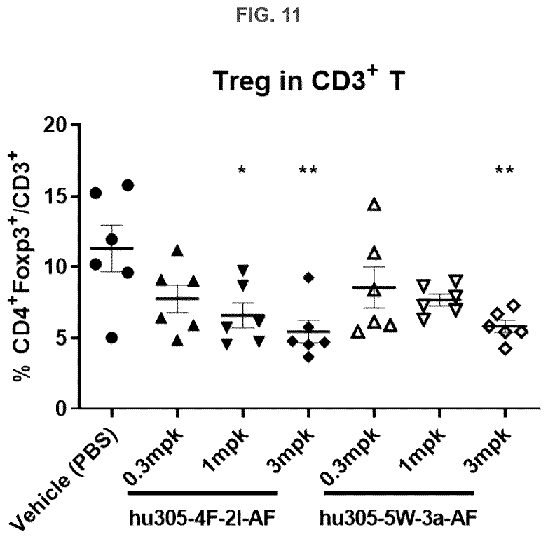

When tumors reached a mean volume of approximately 200-250 mm³ in size, mice were randomized into 7 groups (FIG. 11). After randomization, hu305-4F-21-AF (1 mpk, 3 mpk and 10 mpk) and hu305-5W-3a-AF (1 mpk, 3 mpk and 10 mpk) were injected intraperitoneally (FIG. 11). PBS was administered as vehicle control. Tumors (n=6 mice/treatment group) were harvested on Days 3 post-treatment. Single cells from tumors were dissociated using gentle MACS, followed by filtration using 40 μm cell strainers. After resuspension, cells were counted using a Count star Fluorescence Cell Analyzer. 3×10⁶ cells were then plated in each well of a 96-well plate, and stained for immune cell subsets and functional markers using the flow cytometry antibodies. Antibody fluorescence was detected by flow cytometry on the FACSCelesta™ (BD Biosciences), and the results were analyzed by FlowJo™ software.

The results demonstrated that administration of hu305-4F-21-AF and hu305-5W-3a-AF induced similar extent of intratumoral Treg depletion. Administration of hu305-4F-21 resulted in 31% (1 mpk), 42% (3 mpk), 52% (10 mgk) reduction of Tregs population (% Foxp3+CD4+/CD3+) while administration of hu305-5W-3a resulted in 24% (1 mpk), 32% (3 mpk), 48% (10 mgk) reduction compared to vehicle groups (FIG. 11, Table 22). In summary, both hu305-4F-21-AF and hu305-5W-3a-AF showed potent intratumoral Treg depletion effect in vivo, suggesting the anti-tumor potential.

TABLE 22
Pharmacodynamic Treg depletion effect to anti-CCR8
antibody treatment in MC38 mouse colon cancer model

| Treatment | Dose (mpk) | % Treg Depletion |
|---|---|---|
| PBS | — | NA |
| hu305-4F-2l-AF | 0.1 | 31 |
| hu305-4F-2l-AF | 1 | 42 |
| hu305-4F-2l-AF | 3 | 52 |
| hu305-5W-3a-AF | 0.1 | 24 |
| hu305-5W-3a-AF | 1 | 32 |
| hu305-5W-3a-AF | 3 | 48 |

1. An antibody or antigen-binding fragment thereof which binds to human CCR8 comprising:
(i) a heavy chain variable region that comprises (a) a HCDR1 (Heavy Chain Complementarity Determining Region 1) of SEQ ID NO:14 (b) a HCDR2 of SEQ ID NO:15 and (c) a HCDR3 of SEQ ID NO:6 and a light chain variable region that comprises: (d) a LCDR1 (Light Chain Complementarity Determining Region 1) of SEQ ID NO:7, (e) a LCDR2 of SEQ ID NO:8, and (f) a LCDR3 of SEQ ID NO:9.
(ii) a heavy chain variable region that comprises (a) a HCDR1 (Heavy Chain Complementarity Determining Region 1) of SEQ ID NO:4 (b) a HCDR2 of SEQ ID NO:15 and (c) a HCDR3 of SEQ ID NO:6 and a light chain variable region that comprises: (d) a LCDR1 (Light Chain Complementarity Determining Region 1) of SEQ ID NO:7, (e) a LCDR2 of SEQ ID NO:8, and (f) a LCDR3 of SEQ ID NO:9; or
(iii) a heavy chain variable region that comprises (a) a HCDR1 (Heavy Chain Complementarity Determining Region 1) of SEQ ID NO:4 (b) a HCDR2 of SEQ ID NO:5 and (c) a HCDR3 of SEQ ID NO:6 and a light chain variable region that comprises: (d) a LCDR1 (Light Chain Complementarity Determining Region 1) of SEQ ID NO:7, (e) a LCDR2 of SEQ ID NO:8, and (f) a LCDR3 of SEQ ID NO:9.

2. The antibody or antigen-binding fragment of claim 1, comprising:
(i) a heavy chain variable region (VH) comprising an amino acid sequence at least 90, 91, 92, 93, 94, 95, 96, 97, 98 or 99% identical to SEQ ID NO:10, and a light chain variable region (VL) comprising an amino acid sequence at least 90, 91, 92, 93, 94, 95, 96, 97, 98 or 99% identical to SEQ ID NO: 11;
(ii) a heavy chain variable region (VH) comprising an amino acid sequence at least 90, 91, 92, 93, 94, 95, 96, 97, 98 or 99% identical to SEQ ID NO: 26, and a light chain variable region (VL) comprising an amino acid sequence at least 90, 91, 92, 93, 94, 95, 96, 97, 98 or 99% identical to SEQ ID NO: 23;
(iii) a heavy chain variable region (VH) comprising an amino acid sequence at least 90, 91, 92, 93, 94, 95, 96, 97, 98 or 99% identical to SEQ ID NO: 16, and a light chain variable region (VL) comprising an amino acid sequence at least 90, 91, 92, 93, 94, 95, 96, 97, 98 or 99% identical to SEQ ID NO: 17;
(iv) a heavy chain variable region (VH) comprising an amino acid sequence at least 90, 91, 92, 93, 94, 95, 96, 97, 98 or 99% identical to SEQ ID NO: 16, and a light chain variable region (VL) comprising an amino acid sequence at least 90, 91, 92, 93, 94, 95, 96, 97, 98 or 99% identical to SEQ ID NO: 20; or
(v) a heavy chain variable region (VH) comprising an amino acid sequence at least 90, 91, 92, 93, 94, 95, 96, 97, 98 or 99% identical to SEQ ID NO: 22, and a light chain variable region (VL) comprising an amino acid sequence at least 90, 91, 92, 93, 94, 95, 96, 97, 98 or 99% identical to SEQ ID NO: 23.

3. The antibody or antigen-binding fragment of claim 2, wherein one, two, three, four, five, six, seven, eight, nine, or ten amino acids within SEQ ID NOs: 10 and 11, SEQ ID NOs: 26 and 23, SEQ ID NOs: 16 and 17, SEQ ID NOs: 16 and 20, or SEQ ID NOs: 22 and 23, have been inserted, deleted or substituted.

4. The antibody or antigen-binding fragment of claim 1, that comprises:
(i) a heavy chain variable region (VH) that comprises SEQ ID NO:10, and a light chain variable region (VL) that comprises SEQ ID NO: 11;
(ii) a heavy chain variable region (VH) that comprises SEQ ID NO: 26, and a light chain variable region (VL) that comprises SEQ ID NO: 23;
(iii) a heavy chain variable region (VH) that comprises SEQ ID NO: 16, and a light chain variable region (VL) that comprises SEQ ID NO: 17;
(vi) a heavy chain variable region (VH) that comprises SEQ ID NO: 16, and a light chain variable region (VL) that comprises SEQ ID NO: 20; or
(v) a heavy chain variable region (VH) that comprises SEQ ID NO: 22, and a light chain variable region (VL) that comprises SEQ ID NO: 23.

5. The antibody or antigen-binding fragment of claim 1, which is a monoclonal antibody, a chimeric antibody, a humanized antibody, a human engineered antibody, a single chain antibody (scFv), a Fab fragment, a Fab′ fragment, or a F(ab′)₂ fragment.

6. The antibody or antigen-binding fragment of claim 1, wherein the antibody or antigen-binding fragment thereof has antibody dependent cellular cytotoxicity (ADCC) or complement dependent cytotoxicity (CDC).

7. The antibody or antigen-binding fragment of claim 1, wherein the antibody or antigen-binding fragment thereof has reduced glycosylation or no glycosylation or is hypofucosylated.

8. The antibody or antigen-binding fragment of claim 1, wherein the antibody or antigen-binding fragment thereof has reduced glycosylation or no glycosylation or is afucosylated.

9. The antibody or antigen-binding fragment of claim 1, wherein the antibody or antigen-binding fragment thereof comprises increased bisecting GlcNac structures.

10. The antibody or antigen-binding fragment of claim 1, wherein the antibody or antigen-binding fragment thereof comprises an IgG1 Fc domain.

11. The antibody or antigen-binding fragment of claim 1, which binds to epitopes comprising at least one, two, three, four, five, or six amnio acid residues at positions selected from number 26, 172, 177, 178, 266, and 269 of HuCCR8 amino acid sequence.

12. The antibody or antigen-binding fragment of claim 1, which binds to: (A) epitopes comprising at least one, two or three amnio acid residue(s) at positions selected from number 172, 177, and 269 of HuCCR8 amino acid sequence; or (B) epitopes comprising at least one, two or three amnio acid residues at positions selected from number 26, 178, and 266 of HuCCR8 amino acid sequence.

13. (canceled)

14. The antibody or antigen-binding fragment of claim 1, which binds to: (A) epitopes comprising (1) amino acid residue at position 20-30, (2) amino acid(s) at position 170-180, and (3) amino acid(s) at position 260-270 of HuCCR8 amino acid sequence; or (B) epitopes comprising (1) amino acid residue at position 26, (2) one, two or three amino acid(s) at position 172, 177 and 178, and (3) one or two amino acid(s) at position 266 and 269 of HuCCR8 amino acid sequence.

15. (canceled)

16. The antibody or antigen-binding fragment of claim 1, which binds to epitopes comprising (1) one, two or three amino acid residues at positions in N-terminal, (2) one, two or three amino acid(s) at positions in ECL2 region, and (3) one, two or three amino acid(s) at positions in ECL3 region of HuCCR8 amino acid sequence.

17. A pharmaceutical composition comprising the antibody or antigen-binding fragment thereof, of claim 1 further comprising a pharmaceutically acceptable carrier.

18. A method of treating cancer comprising administering to a patient in need an effective amount of the antibody or antigen-binding fragment of claim 1.

19-26. (canceled)

27. An isolated nucleic acid that encodes the antibody or antigen-binding fragment of claim 1.

28. A vector comprising the nucleic acid of claim 27.

29. A host cell comprising the nucleic acid of claim 27.

30. A process for producing an antibody or antigen-binding fragment thereof comprising cultivating the host cell of claim 29 and recovering the antibody or antigen-binding fragment from the culture.

31-32. (canceled)


SEQUENCE LISTING (29 sequences)

SEQ ID NO: 1 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 2 - protein, 355 aa, synthetic construct
  source 1..355: note = same as monkey_CCR8.pro in Fig. 1A
MDYTLDPSMTTMTDYYYPDSLSSPCDGELIQRNDKLLLAVFYCLLFVFSLLGNSLVILVLVVCKKLRNITDIYLLNLALSDLLFVFSFPFQTYYQLDQWVFGTVMCKVVSGFYYIGFYSSMFFITLMSVDRYLAVVHAVYAIKVRTIRMGTTLSLVVWLTAIMATIPLLVFYQVASEDGVLQCYSFYNQQTLKWKIFTNFEMNILGLLIPFTIFMFCYIKILHQLKRCQNHNKTKAIRLVLIVVIASLLFWVPFNVVLFLTSLHSMHILDGCSISQQLNYATHVTEIISFTHCCVNPVIYAFVGEKFKKHLSEIFQKSCSHIFIYLGRQMPRESCEKSSSCQQHSFRSSSIDYIL

SEQ ID NO: 3 - protein, 353 aa, synthetic construct
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 4 - protein, 5 aa, synthetic construct
SYGVH

SEQ ID NO: 5 - protein, 16 aa, synthetic construct
VIWRGGTTDYNAAFMS

SEQ ID NO: 6 - protein, 16 aa, synthetic construct
NRVTTVVAPNFYAMDY

SEQ ID NO: 7 - protein, 17 aa, synthetic construct
KSSQSLLYSGNQKNYLA

SEQ ID NO: 8 - protein, 7 aa, synthetic construct
WASTRKS

SEQ ID NO: 9 - protein, 9 aa, synthetic construct
QQYYTYPLT

SEQ ID NO: 10 - protein, 124 aa, synthetic construct
QVQMKQSGPSLVQPSQSLSITCTVSGFSLTSYGVHWVRQSPGKGLEWLGVIWRGGTTDYNAAFMSRLSFTKDSSKSQVFFKLNSLQADDTAIYYCAKNRVTTVVAPNFYAMDYWGQGTSVTVSS

SEQ ID NO: 11 - protein, 113 aa, synthetic construct
DIVMTQSPSSLAVSVGEKVTMSCKSSQSLLYSGNQKNYLAWYQQKPGQSPKLLIYWASTRKSGVPDRLTGSGSGTDFTLTISSVKAEDLAAYYCQQYYTYPLTFGAGTRLEIK

SEQ ID NO: 12 - DNA, 372 bp, synthetic construct
  source 1..372: mol_type = other DNA
caggtgcagatgaagcagtcaggacctagcctagtgcagccctcacagagcctgtccataacctgcacagtctctggtttctcattaactagctatggggtacactgggttcgccagtctccaggaaagggtctggagtggctgggagtgatatggagaggtggaaccacagactacaatgcagctttcatgtccagactgagcttcaccaaggacagctccaagagccaagttttctttaaattgaacagtctgcaagctgatgacactgccatatactactgtgccaagaatcgggtaactacggttgtagccccaaatttctatgctatggactactggggtcaaggaacctcagtcaccgtctcgagc

SEQ ID NO: 13 - DNA, 339 bp, synthetic construct
  source 1..339: mol_type = other DNA
gacattgtgatgacccagtctccatcctccctagctgtgtcagttggagagaaggttactatgagctgcaagtccagtcagagccttttatatagtggcaatcaaaagaactacttggcctggtaccagcagaaaccagggcagtctcccaaactgctgatttactgggcatccactaggaaatctggggtccctgatcgcctcacaggcagtggatctgggacagatttcactctcaccatcagcagtgtgaaggctgaagacctggcagcttattactgtcagcaatattatacctatccgctcacgttcggtgctgggaccagactggaaataaaa

SEQ ID NO: 14 - protein, 5 aa, synthetic construct
VYGVH

SEQ ID NO: 15 - protein, 16 aa, synthetic construct
VIWRGGTTDYNPSLKS

SEQ ID NO: 16 - protein, 124 aa, synthetic construct
QVQLQESGPGLVKPSQTLSLTCTVSGFSLAVYGVHWVRQPPGKGLEWLGVIWRGGTTDYNPSLKSRLTFSKDSSKSQVSLKLSSVTAADTAVYYCAKNRVTTVVAPNFYAMDYWGQGTLVTVSS

SEQ ID NO: 17 - protein, 113 aa, synthetic construct
DIVMTQSPDSLAVSLGERVTINCKSSQSLLYSGNQKNYLAWYQQKPGQSPKLLIYWASTRKSGVPDRFSGSGSGTDFTLTISSLQAEDVAAYYCQQYYTYPLTFGQGTRLEIK

SEQ ID NO: 18 - DNA, 372 bp, synthetic construct
  source 1..372: mol_type = other DNA
caagtgcagctgcaagagagcggccccggcctggtgaagcctagccagaccctgagcctgacctgcaccgtgagcggcttcagcctggcagtctacggcgtgcactgggtgagacagccccccggcaagggcctggagtggctgggcgtgatctggagaggcggcaccaccgactacaacccctccctcaagagcagactgaccttcagcaaggacagcagcaagagccaagtgtccctcaagctgagcagcgtgaccgccgccgacaccgccgtgtactactgcgccaagaacagagtgaccaccgtggtggcccccaacttctacgccatggactactggggccaaggcaccctggtgaccgtgagcagc

SEQ ID NO: 19 - DNA, 339 bp, synthetic construct
  source 1..339: mol_type = other DNA
gacatcgtgatgacacagagccccgacagcctggccgtgagcctgggcgagagagtcaccatcaactgcaagagcagccaaagcctgctgtacagcggcaatcagaagaactacctggcctggtatcagcagaagcccgggcagagccccaagctgctgatctactgggcctccacaagaaagagcggcgtgcccgacagattcagcggcagcggcagcggcaccgacttcaccctgaccatcagcagcctgcaagccgaggacgtggccgcgtactactgtcagcagtactacacctaccccctgaccttcggccaaggcacaagactggagatcaag

SEQ ID NO: 20 - protein, 113 aa, synthetic construct
DIVMTQSPDSLAVSLGERVTMNCKSSQSLLYSGNQKNYLAWYQQKPGQSPKLLIYWASTRKSGVPDRFSGSGSGTDFTLTISSLQAEDVAAYYCQQYYTYPLTFGQGTRLEIK

SEQ ID NO: 21 - DNA, 339 bp, synthetic construct
  source 1..339: mol_type = other DNA
gacatcgtgatgacacagagccccgacagcctggccgtgagcctgggcgagagagtcaccatgaactgcaagagcagccaaagcctgctgtacagcggcaatcagaagaactacctggcctggtatcagcagaagcccgggcagagccccaagctgctgatctactgggcctccacaagaaagagcggcgtgcccgacagattcagcggcagcggcagcggcaccgacttcaccctgaccatcagcagcctgcaagccgaggacgtggccgcgtactactgtcagcagtactacacctaccccctgaccttcggccaaggcacaagactggagatcaag

SEQ ID NO: 22 - protein, 124 aa, synthetic construct
QVQLKQSGPGLVKPSETLSLTCTVSGFSLAVYGVHWVRQPPGKGLEWLGVIWRGGTTDYNPSLKSRLTFSKDSSKSQVSLKLSSVTAADTAVYYCAKNRVTTVVAPNFYAMDYWGQGTLVTVSS

SEQ ID NO: 23 - protein, 113 aa, synthetic construct
DIVMTQSPDSLAVSLGERATINCKSSQSLLYSGNQKNYLAWYQQKPGQPPKLLIYWASTRKSGVPDRFSGSGSGTDFTLTISSLKAEDVAVYYCQQYYTYPLTFGQGTRLEIK

SEQ ID NO: 24 - DNA, 372 bp, synthetic construct
  source 1..372: mol_type = other DNA
caagtgcagctgaaacagagcggccccggcctggtgaagcctagcgagaccctgagcctgacctgcaccgtgagcggcttcagcctggcagtctacggcgtgcactgggtgagacagccccccggcaagggcctggagtggctgggcgtgatctggagaggcggcaccaccgactacaacccctccctcaagagcagactgaccttcagcaaggacagcagcaagagccaagtgtccctcaagctgagcagcgtgaccgccgccgacaccgccgtgtactactgcgccaagaacagagtgaccaccgtggtggcccccaacttctacgccatggactactggggccaaggcaccctggtgaccgtgagcagc

SEQ ID NO: 25 - DNA, 339 bp, synthetic construct
  source 1..339: mol_type = other DNA
gacatcgtgatgacacagagccccgacagcctggccgtgagcctgggcgagagagccaccatcaactgcaagagcagccaaagcctgctgtacagcggcaatcagaagaactacctggcctggtatcagcagaagcccgggcagccccccaagctgctgatctactgggcctccacaagaaagagcggcgtgcccgacagattcagcggcagcggcagcggcaccgacttcaccctgaccatcagcagcctgaaagccgaggacgtggccgtgtactactgtcagcagtactacacctaccccctgaccttcggccaaggcacaagactggagatcaag

SEQ ID NO: 26 - protein, 124 aa, synthetic construct
QVQLKQSGPGLVKPSETLSLTCTVSGFSLTSYGVHWVRQPPGKGLEWLGVIWRGGTTDYNPSLKSRVTFSKDSSKNQVSLKLSSVTAADTAVYYCAKNRVTTVVAPNFYAMDYWGQGTLVTVSS

SEQ ID NO: 27 - DNA, 372 bp, synthetic construct
  source 1..372: mol_type = other DNA
caagtgcagctgaaacagagcggccccggcctggtgaagcctagcgagaccctgagcctgacctgcaccgtgagcggcttcagcctgacaagctacggcgtgcactgggtgagacagccccccggcaagggcctggagtggctgggcgtgatctggagaggcggcaccaccgactacaacccctccctcaagagcagagtgaccttcagcaaggactccagcaagaaccaagtgtccctcaagctgagcagcgtgaccgccgccgacaccgccgtgtactactgcgccaagaacagagtgaccaccgtggtggcccccaacttctacgccatggactactggggccaaggcaccctggtgaccgtgagcagc

SEQ ID NO: 28 - protein, 264 aa, synthetic construct
MDYTLDLSVTTVTDYYYPDIFSSPSDAELIQTNGKGGGGSCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 29 - protein, 262 aa, synthetic construct
MDYTLDLSVTTVTDYYYPDIFSSPSDAELIQTNGKGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK